(19) United States
(12) Patent Application Publication
(10) Pub. No.: US 2020/0011856 A1
(43) Pub. Date: Jan. 9, 2020

(54) Method for Determining the Effectiveness of Treatment for the Prevention of Heartworm Disease

(71) Applicants: BOEHRINGER INGELHEIM ANIMAL HEALTH USA INC., DULUTH, GA (US); THOMAS JEFFERSON UNIVERSITY, PHILADELPHIA, PA (US)

(72) Inventors: Brenda BONDESEN, Indianapolis, IN (US); John Matthew HARRINGTON, Athens, GA (US); David ABRAHAM, Wynnewood, PA (US); Jessica HESS, Willow Grove, PA (US)

(73) Assignees: BOEHRINGER INGELHEIM ANIMAL HEALTH USA INC., DULUTH, GA (US); THOMAS JEFFERSON UNIVERSITY, Philadelphia, PA (US)

(21) Appl. No.: 16/484,768

(22) PCT Filed: Feb. 8, 2018
(86) PCT No.: PCT/US2018/017398

§ 371 Date: Aug. 8, 2019

Related U.S. Application Data
(60) Provisional application No. 62/456,510, filed on Feb. 8, 2017.

(51) Int. Cl. G01N 33/50; A01K 67/027

(52) U.S. Cl. CPC G01N 33/5085; A01K 67/0271; A01K 2207/12; A01K 2267/0337; A01K 2227/105; G01N 2333/4353

(57) ABSTRACT

The following discloses a novel model for parasitic worm infection, which is useful for screening for and evaluating the efficacy of antiparasitic agents. In particular, the disclosure provides an immunocompromised mouse model that, for the first time, supports advanced development of Dirofilaria immitis (Di) parasites outside of their definitive hosts. As described herein, early-stage larval forms of Di, the causative agent of Canine Heartworm disease, not only persist in this model, but they develop into mature worms.

### CROSS-REFERENCE TO RELATED APPLICATIONS

This application claims the benefit of U.S. provisional patent application No. 62/456,510, filed Feb. 8, 2017, the disclosure of which is herein incorporated by reference in its entirety.

### FIELD OF THE INVENTION

The invention relates to the modeling of parasitic worm infections using immunocompromised rodents, with or without engrafted immune cells from a definitive host of the parasitic worm. The invention further relates to the use of such model systems to screen and evaluate the efficacy of antiparasitic agents.

### BACKGROUND OF THE INVENTION

Filariasis is a parasitic disease caused by thread-like filarial nematodes or roundworms. Filariasis is transmitted via insect bites, and infective filarial larvae may be introduced into animals, including dogs and humans, via bites of blood sucking insects including mosquitoes or flies. In dogs, cardiopulmonary dirofilariasis, (i.e., heartworm (HW) disease) is caused by Dirofilaria immitis (D. immitis) and Dirofilaria repens (D. repens) nematodes. In the case of D. repens, adults are subcutaneous, so while they don't cause heartworm disease per se, they do cause disease/pathology and are prevented with IVM doses comparable to those used to prevent HW. Dirofilariasis is a severe vector-borne helminthiasis of dogs and cats worldwide. It is transmitted by Culicidae (i.e., mosquito) bites and caused by the development of D. immitis in the pulmonary arteries and the heart. Clinically, it leads to irreversible cardiac insufficiency that can result in the death of the animal. Treatment is difficult and prevention is critical for dirofilariasis control. In humans, clinically significant filarial nematodes include Wuchereria bancrofti, Brugia malayi, Brugia timori, Onchocerca volvulus and Mansonella species. W. bancrofti and B. malayi infections can develop into lymphatic filariasis, often presenting as hydrocoele and/or lymphedema, and (in extreme cases), elephantiasis. O. volvulus infections can evolve into onchocerciasis, the visual impairment giving the disease its common name River Blindness. The nematodes are host to obligate intracellular bacteria of the genus Wolbachia. These endobacteria are essential for embryogenesis, larval development and adult worm survival. As such, an alternative effort aiming to eliminate the nematodes includes removal of the bacteria with antibacterial agents.

The development of these types of parasitic worms, particularly Dirofilaria, is often highly restricted to the definitive (mammalian) host, precluding the development of intermediate animal/rodent models. The ramifications of this are apparent in the lack of new pharmaceutical agents to combat these pathogens. For example, macrocyclic lactones (MLs) were introduced more than 25 years ago, yet they remain the only chemical class approved for HW disease prevention. For most investigators, canine studies are simply too long—at least six months—and expensive to justify exploratory studies outside this proven class of compounds. Therefore, an intermediate model of filarial infection in rodents could open the discovery floodgates, allowing researchers to rapidly screen and evaluate novel parasiticidal agents. As of the filing of this application, no one has successfully modeled such filarial infections outside the definitive host.

### SUMMARY OF THE INVENTION

Applicants have found, quite unexpectedly, that NOD scid gamma (NOD.Cg-Prkdcscid Il2rgtm1Wj1/SzJ; hereafter, “NSG”) immunodeficient mice support complete infection and development of certain parasitic worms, including canine HW. These mice support complete infection and development of filarial parasitic worms, such as those in Table 1, as well as other parasitic nematodes, such as Haemonchus contortus. Additionally, Applicants discovered that NRG (NOD-Rag1^{null} IL2rg^{null}, NOD rag gamma), a similarly immunodeficient mouse, is susceptible to HW, whereas other immunodeficient mice are resistant.

TABLE 1
Immunocompromised mouse unexpectedly supports
complete infection for some nematode species, but not others.

| | | Complete | Complete |
|---|---|---|---|
| | Definitive | Infection in | Infection in |
| Parasite | Host | Normal Mouse? | NSG Mouse? |
| Dirofilaria immitis | Canine | No | Yes |
| (Heartworm Disease) | | | |
| Onchocerca volvulus | Human | No | No |
| (River Blindness) | | | |
| Strongyloides stercoralis | Human, | No | Yes |
| | Canine | | |
| Haemonchus contortus | Sheep, | No | Yes |
| | goats | | |

Accordingly, in a first aspect, the invention provides an immunocompromised rodent model for supporting the infection and development of parasitic worms (e.g., nematode parasites such as filarial parasites) outside of their definitive hosts. Such immunocompromised rodent models can support the complete infection and development of filarial parasites (e.g., to the stage of adult worms) outside of their definitive hosts. Such immunocompromised rodent models can be used in methods for modeling complete infection of a parasitic worm outside of the worm's definitive host, methods of determining the effectiveness of a compound for treating or preventing a parasitic worm infection, methods of drug screening for drugs with efficacy in treating or preventing a parasitic worm infection, or methods of characterizing changes in sensitivity to a parasiticidal agent during parasitic worm infection (e.g., a filaricidal agent during filarial worm infection).

In a second aspect, the invention provides for methods of using the immunocompromised rodent model described above for modeling complete infection of a parasitic worm or nematode outside of the worm's definitive host.

In a third aspect, the invention provides for methods of using the immunocompromised rodent model to evaluate the efficacy of known or suspected parasiticidal, including filaricidal and microfilaricidal, compounds.

In a fourth aspect, microfilariae may be administered to the immunocompromised rodents, to allow for the screening and evaluation of APIs that selectively target this earlier stage of the filarial parasites.

In a fifth aspect, the invention provides for methods of treating parasitic worm infections (e.g., nematode parasite infections), comprising administering to a subject in need thereof an effective amount of a parasiticidal compound identified using the immunocompromised rodent model as described in the third or fourth aspect.

These and other embodiments are disclosed or are obvious from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 is a graph showing the growth of D. immitis recovered from NSG mice at the indicated times post-infection with 100 L3 per NSG mouse.

FIG. 2 is a graph showing the normalized percent larva recovery (day 42/6 weeks) after D. immitis (Di)-infected NSG mice were dosed with 1 mg/kg emodepside or 3 mg/kg ivermectin, at Days 1, 15, and 30 post-infection.

FIG. 3 is a graph showing the lengths of Di recovered at Days 41 and 43, post-infection, from NSG mice that were administered Control, EMO, or 8800 on Day 30, post-infection.

FIG. 4 is a graph showing the lengths of Di recovered at Days 83 and 84. Control, EMO, or 8800 treatments was administered to the Di-infected mice on Days 30 and 60.

FIG. 5 is a graph showing the lengths of Di recovered at Days 41 to 43. Control and various concentrations of 8800, EMO, or IVM was administered to the Di-infected mice on Days 30 and 60.

FIG. 6 is a graph showing length of Di recovered at Days 41 to 43. Control, EMO, 8800, 4838, 8914, or 6557 was administered to the Di-infected mice on Days 1, 15, and 30 post-infection.

FIG. 7 is a graph showing the lengths of Di recovered at Days 41 to 43. Control, EMO, 8800, 4838, or IVM was administered to the Di-infected mice on Days 1, 15, and 30 post-infection.

FIG. 8 is a graph showing the lengths of Di recovered at Days 41 to 42. Control, EMO, 8800, or 4838 was administered to the Di-infected mice on Days 1, 15, and 30 post-infection.

FIG. 9 is a graph showing the lengths of Di recovered at Days 41 to 42. Control, EMO, 8800, or 4838 was administered to the Di-infected mice on Days 1, 15, and 30 post-infection.

FIG. 10 is a graph showing the lengths of Di recovered at Days 40 to 42. Control, EMO, 8800, or 4838 was administered to the Di-infected mice on Days 1, 15, and 30 post-infection.

FIG. 11 is a graph showing the lengths of Di recovered at Days 41 to 42. Control, IVM, 8800, or 4838 was administered to the Di-infected mice on Days 1, 15, and 30 post-infection.

FIG. 12 is a graph showing the lengths of Di recovered at Days 41 to 42. Control or IVM was administered to the Di-infected mice on Days 1, 15, and 30 post-infection. The Missouri and JYD strains of Di were tested.

FIG. 13 shows worm recovery locations in Di-infected mice administered control or IVM on Days 1, 15, and 30 post-infection.

FIG. 14 is a graph showing the lengths of Di recovered at Days 41 to 42 post-infection from C57BL/6, SCID, NOD, NOD/SCID, NSG, and NRG mice.

FIG. 15 is a graph showing the lengths of Di recovered at Days 41 to 42. Control or 4838 was administered to the Di-infected mice on Days 1, 15, and 30 post-infection.

FIG. 16 is a graph showing the lengths of Di recovered at Days 41 to 42. Control, 3615, 5010, 7823, 2677, or 8800 was administered to the Di-infected mice on Days 1, 15, and 30 post-infection.

FIG. 17 is a graph showing the lengths of Di recovered at Days 40 to 42. Control or IVM was administered to the Di-infected mice on Days 1, 15, and 30 post-infection.

FIG. 18 is a graph showing the lengths of Di recovered at Days 40 to 42. Control or 7823 was administered to the Di-infected mice on Days 1, 15, and 30 post-infection.

FIG. 19 is a graph showing the lengths of Di recovered at Days 40 to 42. Control or IVM was administered to the Di-infected mice on Days 1, 15, and 30 post-infection.

FIG. 20 is a graph showing the lengths of Di recovered at Days 40 to 42. Control or IVM was administered to the Di-infected mice on Days 1, 15, and 30 post-infection.

FIG. 21 is a graph showing the lengths of Di recovered at Days 40 to 42 post-infection from mice with engrafted canine CD34+ cord blood cells.

FIG. 22 is a graph showing the number of worms recovered in control and MPA-treated NSG mice infected with 500 L3 H. contortus.

FIG. 23 is a graph showing the number of worms recovered in control and MPA-treated NSG mice infected with 1000 L3 H. contortus.

### DETAILED DESCRIPTION

The invention is based upon the surprising discovery that immunocompromised rodents, with or without engrafted immune cells from a different species (e.g., the species of a definitive host of a parasitic worm), may be used to model filarial parasite infections and infections by other parasitic worms such as nematode parasites outside of their definitive hosts. Applicants have found, quite unexpectedly, that NSG and NRG immunodeficient mice support infection and development of certain parasitic worms, including canine HW.

NSG and NRG mice are among the most immunocompromised mice described to date, lacking mature T cells, B cells, and natural killer (NK) cells. Moreover, NSG mice lack several cytokine signaling pathways, and they have many defects in innate immunity. As indicated in Table 1, Applicants found that NSG and NRG mice exhibit a surprising capacity to harbor parasites that until now have normally only established complete infections in definitive hosts, but have not established complete infections in non-target animals (i.e., non-definitive hosts) having uncompromised immune systems (Table 1). Likewise, Applicants found that NSG mice with engrafted immune cells from the definitive host of a parasitic worm exhibit a surprising capacity to harbor parasites that until now have normally only established complete infections in definitive hosts. As used herein, “complete infection” means that a given early stage larval form (e.g., L3) persists and develops into mature worms (e.g., adult worms). In the case of filarial parasites, the given early stage larval form (e.g., L3) persists and develops into mature worms that may be capable of releasing microfilariae into the bloodstream of the infected/infested host. Microfilariae themselves do not establish infection. Instead, these earliest stage larval forms are taken up from an infected mammal via a bite from a blood-sucking arthropod (e.g., mosquito). Within the vector, microfilariae develop into L3 larvae, which are introduced into subsequent mammalian hosts when an L3-infected vector bites and feeds upon the mammal.

This finding is unexpected, in part, because a skilled person would not have held a reasonable expectation that L3 larvae would persist in a non-definitive host for the observed long (months) periods of time. As such, prior to this disclosure, it could have been the case that the immunocompromised mouse was good for harboring the L3 stage, but only for a limited period of time. Such a model might have been sufficient to test therapeutic agents for their ability to kill L3 larvae. However, much to Applicants' surprise, the L3 larvae developed into L4 and adults when introduced into the immunocompromised rodent. In the case of Dirofilaria immitis, for example, this provides an excellent model for canine cardiopulmonary dirofilariasis. Applicants' establishment of this model permits the rapid screening of APIs for their ability to target one or more specific stages of a filarial nematode's live cycle. Applicants' establishment of this model also permits the rapid screening of APIs for their ability to target one or more specific stages of the life cycle of other parasitic worms or nematode parasites. This feature has the obvious benefit of allowing investigators to target parasite life cycle stages that were heretofore extremely expensive and challenging to address because of the requirement for a definitive host. Moreover, Applicants envision that this model will provide an invaluable tool for interrogating the nature of the signals that allow immunocompromised rodents to mimic canine filarial infection and development. Finally, Applicants envision that when microfilariae are introduced into the immunocompromised rodents, they may persist for a period of time, particularly in circulation when introduced via the IV route. Such persistence would allow for the testing of APIs that are specifically active against microfilariae in vivo. Likewise, Applicants envision that when infective larvae (e.g., L3) of nematode parasites are introduced into the immunocompromised rodents, they may persist for a period of time and, for example, develop into adult worms, allowing for API testing.

In a first aspect, the invention provides an immunocompromised rodent model for supporting the complete infection and development of parasitic worms (e.g., to a mature stage such as an adult worm) outside of their definitive hosts. For example, the immunocompromised rodent model can support the complete infection and development of filarial parasites (e.g., to adult filarial worms) outside of their definitive hosts. In some embodiments, the rodent is a rat or a mouse. In some embodiments, the rodent is a mouse. Such immunocompromised rodent models can be used in methods for modeling complete infection of a parasitic worm outside of the worm's definitive host, methods of determining the effectiveness of a compound for treating or preventing a parasitic worm infection, methods of drug screening for drugs with efficacy in treating or preventing a parasitic worm infection, or methods of characterizing changes in sensitivity to a parasiticidal agent during parasitic worm infection (e.g., a filaricidal agent during filarial worm infection).

In some embodiments, the model comprises infection of a mouse lacking mature or functional T cells, B cells, and natural killer (NK) cells. In some embodiments, the model comprises infection of a mouse lacking mature T cells, B cells, and natural killer (NK) cells; and, wherein the mouse also lacks several cytokine signaling pathways and comprises many defects in innate immunity. For example, the model can comprise an NSG mouse or an NRG mouse as described elsewhere herein or can be genetically engineered to lack one or more or all comparable functional elements that are lacking in an NSG mouse, relative to a corresponding mouse possessing an intact immune system.

NSG (NOD.Cg-Prkdc^{scid}Il2rg^{tm1Wj1}/SzJ) is one of the most immunodeficient mouse strains. The Non-Obese Diabetic (NOD) genetic background contains alleles that reduce the function of the innate branch of the immune system. Consequently, macrophages and dendritic cells are defective. Scid is a loss-of-function mutation of the Prkdc gene that prevents the development of T and B cells. Prkdc encodes the catalytic subunit of a DNA dependent protein kinase with a role in resolving the DNA double strand breaks that occur during V(D)J recombination (i.e., via non-homologous end joining (NHEJ)). In the absence of V(D)J recombination, the T cell receptor (TCR) gene in T cells and the immunoglobulin (Ig) gene in B cells are not expressed, and T and B cells cannot mature. Due to the NHEJ defect in scid mutants, scid mice lack both mature T and B cells. The gamma chain of the interleukin 2 receptor (Il2rg) is a common component of the cell surface receptors for six different interleukins (IL-2, IL-4, IL-7, IL-9, IL-15, and IL-21). NSG mice have a complete null mutation (knockout) of this gene. The signaling pathways for these cytokines are blocked in Il2rg knockout mice, although the cytokines themselves are still present. The major consequence of Il2rg deficiency is an absence of functional NK cells, which require IL15 signaling to develop.

NSG mice have the following characteristics: mature B cells: absent; mature T cells: absent; dendritic cells: defective; macrophages: defective; natural killer cells: absent; and complement: absent.

NSG and NRG (NOD rag gamma; NOD.Cg-Rag1^{tm1Mom} Il2rg^{tm1Wj1}/SzJ; NOD Rag1^{null} Il2rg^{null}) mice are very similar strains. NRG mice substitute the Rag1 knockout mutation for the scid mutation. The Rag1 knockout has a very similar phenotype in the immune system (elimination of T and B cells), but it does not have the scid side effect of radiation/chemotherapy sensitivity. NRG mice carry two mutations on the NOD/ShiLtJ genetic background: a targeted knockout mutation in recombination activating gene 1 (Rag1), and a complete null allele of the IL2 receptor common gamma chain (Il2rg). The Rag1^{null} mutation renders the mice B and T cell deficient, and the IL2rg^{null} mutation prevents cytokine signaling through multiple receptors, leading to a deficiency in functional NK cells.

In other embodiments, the model comprises a mammal whose immune system has been genetically engineered to lack comparable functional elements that are lacking in an NSG mouse, relative to a corresponding mouse possessing an intact immune system. Such models can have, for example, one or more or all of the following characteristics: (1) absence of functional T and B cells (i.e., lack of mature T and B cells); (2) absence of functional NK cells; and (3) reduced function of the innate branch of the immune system. Some models lack mature T and B cells. This can be caused, for example, by a loss of function mutation in the Prkdc gene, such as the Scid mutation. This can also be caused, for example, by a loss of function mutation in the Rag1 gene. Some models lack functional NK cells. This can be caused, for example, by a loss-of-function mutation in the Il2rg gene, which can cause deficient IL15 signaling. Some models have deficient cytokine production, such as deficient interleukin signaling pathways (e.g., those affected by Il2rg knockout, such as IL15 signaling). Some models have reduced function of the innate branch of the immune system. For example, macrophages and/or dendritic cells can be defective in some models. This can be caused, for example, by the NOD genetic background. Some models lack mature T and B cells and lack functional NK cells. Some models lack mature T and B cells and have reduced function of the innate branch of the immune system. Some models lack functional NK cells and have reduced function of the innate branch of the immune system. Some models lack mature T and B cells, lack functional NK cells, and have reduced function of the innate branch of the immune system.

In some embodiments, the model comprises a mammal whose immune system has been genetically engineered to be permissive to complete infection by parasitic worms, including filarial worms, including HW. In some embodiments, the mammal may comprise engrafted immune cells from another species, such as engrafted immune cells from a definitive host of a particular parasitic worm. In some embodiments, the mammal may comprise engrafted hematopoietic stem cells. In some embodiments, the mammal may comprise engrafted mature immune cells. In some embodiments, the mammal may comprise engrafted canine stem cells. In other embodiments, the mammal may comprise engrafted canine mature immune cells. Such mammals may lack functional NK cells and/or lack mature T and B cells (e.g., have the scid mutation). In some cases, the mammal can be irradiated prior to engraftment. The mammal can be a rodent, such as a rat or a mouse. For example, the severe immunodeficiency of NSG and NRG mice allows them to be caninized by engraftment of canine CD34+ hematopoietic stem cells (HSCs) and canine derived xenografts at high efficiency. Such engrafted mammals may carry canine immune cells that have either been generated in the mammal (e.g., following CD34+ canine hematopoietic stem cell injections) or generated in a canine donor and injected into the mammal (PBMCs). CD34 is a marker for stem/progenitor cells that are capable of producing every hematopoietic lineage. When injected in an immunocompromised mammal (e.g., a NSG mouse), they naturally migrate to the bone marrow and differentiate into the mature cell types of the immune system, along the established progenitor pathways. CD34+ cells can be readily isolated from mobilized peripheral blood, umbilical cord blood, or bone marrow. PBMCs (“peripheral blood mononuclear cells”) include mature lymphocytes (B, T, and NK cells), monocytes, and macrophages. When injected in the immunocompromised mammal (e.g., a NSG mouse), PBMCs either remain in circulation (e.g., T cells), or die/migrate to other tissues (e.g., other cell types).

The parasitic worm in the above models can be, for example, a nematode parasite such as a filarial worm or a gastrointestinal parasite. The nematode parasite in the above models can be, for example, from the Spirurida order or the Filarioidea superfamily. Species within this superfamily are known as filarial worms or filariae (singular: filaria). Infections with parasitic filarial worms cause disease conditions generically known as filariasis. Filarial parasites and their life cycle are described in more detail elsewhere herein. They are vectored by arthropods, mature and mate in specific host tissues, and produce microfilariae. The Filarioidea include several families, such as Aproctidae, Creagrocercidae, Drilonematidae, Filariidae, Homungellidae, Mesidionematidae, Onchocercidae, Scolecophilidae, Setariidae, and Ungellidae. Examples of genera within these families include Wuchereria, Brugia, Onchocerca, Mansonella, Dirofilaria, Parafilaria, Stenofilaria, and Loa. Examples of filarial parasites include the following: Wuchereria bancrofti; Brugia species (e.g., Brugia malayi, and Brugia timori); Mansonella species (e.g., Mansonella ozzardi, Mansonella perstans, Mansonella streptocerca, and Mansonella semiclarum or Mansonella perstans); Dirofilaria immitis; Dirofilaria repens; Parafilaria species (e.g., Parafilaria bovicola and Parafilaria multipapillosa); Onchocerca species (e.g., Onchocerca volvulus, Onchocerca dermata, Onchocerca ochengi, and Onchocerca dukei); Stenofilaria assamensis; and Loa loa.

In another example, the nematode parasite can be a gastrointestinal parasite. A gastrointestinal parasite refers to a parasite that infects the gastrointestinal tract. For example, the nematode can be from the Strongylida order or the Trichostrongyloidea superfamily (e.g., the Haemonchus genus). Trichostrongyloidea are divided into 14 families and 24 subfamilies. These worms have very small mouths and are found in a large number of hosts. They generally infect the stomach or intestine. One example of such a parasite is Haemonchus contortus (barber's pole worm), which is described in more detail elsewhere herein. Adult worms attach to the abomasal mucosa and feed on blood. The disease caused by Haemonchus worms is called haemonchosis. In another example, the nematode parasite can be from the Rhabditida order (e.g., the Strongyloididae family or the Strongyloides genus). For example, the nematode parasite can be Strongyloides stercoralis (threadworm), which is a human pathogenic parasitic roundworm causing the disease strongyloidiasis. The adult parasitic stage lives in tunnels in the mucosa of the small intestine. S. stercoralis has been reported in other mammals, including cats and dogs.

In a second aspect, the invention provides for methods of using the immunocompromised rodent model described above for modeling complete infection of a parasitic worm or nematode outside of the worm's definitive host. Such methods can comprise, for example, infecting an immunocompromised rodent with a specific number of worm larvae such that one or more of the worm larvae persist and develop into mature worms.

In a third aspect, the invention provides for methods of using the immunocompromised rodent model described above to evaluate the efficacy of known or suspected parasiticidal compounds. Such methods can determine the effectiveness of a treatment (e.g., a compound, such as a small molecule or a biologic) for treating or preventing a parasitic worm infection (i.e., in the definitive host of the parasitic worm). For example, such methods can be drug screening methods comprising administering putative or known parasiticidal compounds or biologics to any of the rodent models described above. Also provided are methods of characterizing changes in sensitivity to a parasiticidal (e.g., filaricidal) agent during parasitic worm (e.g., nematode or filarial worm or gastrointestinal parasite) infection using any of the rodent models described above. Also provided are methods of drug screening (e.g., for drugs with activity against a parasitic worm infection).

As one example, such methods can comprise infecting an immunocompromised rodent with a specific number of parasitic worm larvae, treating the infected rodent with a compound or a combination of compounds to be tested, and necropsying the rodent and determining the number of parasitic worms in a rodent tissue (e.g., an infected rodent tissue). As another example, such methods can comprise determining the number of parasitic worms in one or more isolated tissue samples (e.g., isolated infected tissue samples) of an immunocompromised rodent, preferably necropsied immunocompromised rodent, infected with a specific number of parasitic worm larvae and treated with a compound or combination of compounds to be tested, wherein such method comprises determining the number of parasitic worms presented in such isolated tissue samples. By infected tissue is meant a tissue in which that type of worm is found in an infected, but untreated immunocompromised rodent.

The worms counted in the tissue can be any stage of worm (e.g., microfilariae (Mf), L3, L4, L5, or adult worms). Likewise, eggs can be counted. In one embodiment, the number of adult worms is counted. Preferably, the tissue in which the worms are counted is a tissue in which that type of worm is typically found in an infected definitive host or a tissue in which that type of worm is found in an infected, but untreated immunocompromised rodent. For example, for D. immitis, the tissue can be muscle or skin. As another example, the tissue for a gastrointestinal parasite such as Strongyloides stercoralis or Haemonchus contortus can be the stomach, the small intestine, or another tissue from the gastrointestinal tract.

The worms can be counted at different time points post-infection. For example, the worms can be counted at about 1, 2, 3, 4, 5, 6, 7, 8, 9, 10, 11, 12, 13, 14, 15, or more weeks post-infection. In some embodiments, the worms are counted at about 2-10, 3-9, 4-8, 5-7, 6 weeks post-infection.

The rodents used in such methods in the second and third aspects can be any of the rodent models described above. In some embodiments, the rodents are rats or mice. In some embodiments, the immunocompromised rodent model is characterized by the absence of mature T and B cells and a lack of functional NK cells. In some embodiments, the immunocompromised rodent model is characterized by a deficiency in cytokine signaling and multiple defects in innate immunity. In some embodiments, the immunocompromised rodent model is further characterized by the absence of production of detectable serum immunoglobulin. In some embodiments, the rodent has been genetically engineered to disrupt or delete genes required for wild type immune function. In some embodiments, the rodent has a Prkdc gene comprising a severe combined immunodeficiency (scid) mutation. In some embodiments, the rodents are NSG or NRG mice. In some embodiments of the second aspect, the rodents are NSG mice.

In some embodiments, the rodents in the methods of the second or third aspect are genetically-engineered to lack all the immune system elements that are necessary and sufficient to support complete infection of a parasitic worm that does not develop in a rodent having an intact immune system. Such models can have, for example, one or more or all of the following characteristics: (1) absence of functional T and B cells (i.e., lack of mature T and B cells); (2) absence of functional NK cells; and (3) reduced function of the innate branch of the immune system. Some models lack mature T and B cells. This can be caused, for example, by a loss of function mutation in the Prkdc gene, such as the Scid mutation. This can also be caused, for example, by a loss of function mutation in the Rag1 gene. Some models lack functional NK cells. This can be caused, for example, by a loss-of-function mutation in the Il2rg gene, which can cause deficient IL15 signaling. Some models of deficient cytokine production, such as deficient interleukin signaling pathways (e.g., those affected by Il2rg knockout, such as IL15 signaling). Some models have reduced function of the innate branch of the immune system. For example, macrophages and/or dendritic cells can be defective in some models. This can be caused, for example, by the NOD genetic background. Some models lack mature T and B cells and lack functional NK cells. Some models lack mature T and B cells and have reduced function of the innate branch of the immune system. Some models lack functional NK cells and have reduced function of the innate branch of the immune system. Some models lack mature T and B cells, lack functional NK cells, and have reduced function of the innate branch of the immune system.

In some embodiments, the method of the second or third aspect may be practiced using a rodent whose immune system has been genetically engineered to lack only the elements that are necessary for it to be permissive to complete infection by (and development of) parasitic worms, including filarial worms, including D. immitis. In such embodiments, some elements of the immune system that are lacking and/or deficient in NSG mice need not be deficient in the engineered mouse for said engineered mouse to support complete infection and development by the parasitic worm.

In some embodiments, the immunocompromised rodent used in the methods of the second or third aspect comprises engrafted immune cells. The engrafted immune cells can be from another species, such as engrafted immune cells from a definitive host of a particular parasitic worm. In some embodiments, the immunocompromised rodent may comprise engrafted hematopoietic stem cells. In some embodiments, the immunocompromised rodent may comprise engrafted mature immune cells. For example, the immunocompromised rodent can comprise engrafted canine immune cells. The engrafted immune cells can be CD34+ cells or hematopoietic stem cells (e.g., from umbilical cord blood or bone marrow). For example, about 10,000, 20,000, 30,000, 40,000, 50,000, 60,000, 70,000, 80,000, 90,000, 100,000, 10,000-100,000, 20,000-100,000, 30,000-100,000, 40,000-100,000, or 50,000-100,000 or more CD34+ cells can be injected into the immunocompromised rodent model. Alternatively, the engrafted immune cells can be mature immune cells, such as PBMCs (“peripheral blood mononuclear cells”) including, for example, mature lymphocytes (B, T, and NK cells), monocytes, and macrophages. For example, about 1×10⁶, 2×10⁶, 3×10⁶, 4×10⁶, 5×10⁶, 6×10⁶, 7×10⁶, 8×10⁶, 9×10⁶, 10×10⁶, 15×10⁶, 20×10⁶, 5×10⁶-10×10⁶, 1×10⁶-20×10⁶, 2×10⁶-20×10⁶ 3×10⁶-20×10⁶, 4×10⁶-20×10⁶, or 5×10⁶-20×10⁶ or more PBMCs can be injected into the immunocompromised rodent.

Examples of parasitic worms that can be used in the immunocompromised rodent models in the methods of the second or third aspect are described in more detail above. The parasitic worm can be, for example, a nematode. The nematode in the above models can be, for example, a filarial worm. In one embodiment, the filarial worm is Dirofilaria immitis. Other nematodes described elsewhere herein can also be used. For example, gastrointestinal parasites such as Haemonchus contortus and Strongyloides stercoralis can be used.

The immunocompromised rodent can be infected in the methods of the second or third aspect by any suitable means. Examples of suitable means include subcutaneous injection, intraperitoneal injection, and oral administration. For example, for filarial worms such as D. immitis, the infection can be through administration of the larvae by subcutaneous injection or intraperitoneal injection. For gastrointestinal parasites, the infection can be, for example, through administration of the larvae by oral gavage.

The number of parasitic worm larvae used to infect the immunocompromised rodent in the methods of the second or third aspects can be any number sufficient to establish a complete infection. For example, about 5, 10, 15, 20, 25, 30, 35, 40, 45, 50, 60, 70, 80, 90, 100, 110, 120, 130, 140, 150, 200, 300, 400, 500, 1000, 2000, 3000, 4000, 5000, 25-5000, 6000, 7000, 8000, 9000, 10000, 1000-10000, 2000-9000, 3000-8000, 4000-7000, 5000-6000, 1000-5000, 5000-10000, 25-4000, 25-3000, 25-2000, 25-1000, 100-5000, 200-4000, 300-3000, 400-2000, 500-1000, 25-500, 50-200, or 50-100 parasitic worm larvae can be used. In one embodiment, about 50-100 parasitic worm larvae (e.g., D. immitis larvae) are used. In one embodiment, 500-1000 parasitic worm larvae (e.g., Haemonchus contortus larvae) are used. In one embodiment, 1000-5000 parasitic worm larvae (e.g., Strongyloides stercoralis) are used. The parasitic worm larvae can be any stage, such as any stage sufficient to infect the rodent. For example, the stage of parasitic worm (e.g., nematode or filarial worm) used can be microfilariae, L3 larvae, L4 larvae, L5 larvae, or young adult worms. In one embodiment, L3 larvae are used.

In some embodiments, the method of the third aspect can further comprise infecting a control immunocompromised rodent with a specific number of parasitic worm larvae, necropsying the control immunocompromised rodent and determining the number of worms present in a rodent tissue, and comparing the number of worms in the rodent tissue from the control (i.e., untreated) immunocompromised rodent with the number of worms in the rodent tissue from the treated immunocompromised rodent. Preferably, the control and the treated rodents are infected with the same number of parasitic worm larvae by the same means of infection. Preferably, the tissue samples from the control and the treated rodents are the same type of tissue isolated at the same time point post-infection.

In some embodiments, the method of the third aspect can further comprise comparing the parasiticidal efficacy of the test compound(s) against the parasiticidal efficacy of a compound known to have parasiticidal activity against the parasitic worm being tested. For example, the method can further comprise comparing the parasiticidal efficacy of the test compound(s) against the parasiticidal efficacy of either or both of a macrocyclic lactone (ML) parasiticidal compound or a depsipeptide parasiticidal compound. Examples of ML parasiticidal compounds include avermectin, ivermectin, doramectin, abamectin, milbemycin, and moxidectin. Examples of depsipeptide parasiticidal compound include emodepside.

In some embodiments, the efficacy of putative parasiticidal compounds may be tested against a target parasite up to about 95% faster than efficacy studies in dogs or other definitive host animals. In some embodiments, the efficacy of putative filaricidal and/or microfilaricidal compounds may be tested against a target parasite up to about 95% faster than efficacy studies in dogs. In some embodiments, emodepside and ivermectin eliminate HW parasites when said active pharmaceutical ingredients (APIs) were delivered orally to HW-infected NSG mice. To Applicants' knowledge, the data disclosed herein are the first to demonstrate that any immunocompromised rodent is capable of harboring, and promoting the development of, HW.

As used herein, a “parasiticidal” agent is one that kills parasitic worms at any stage of development, unless otherwise specified. As used herein, a “filaricidal” agent is one that kills filarial worms at any stage of development, unless otherwise specified. Similarly, a “microfilaricidal” agent is one that kills microfilariae. In some cases, an agent may be described as being “selective” for one or more stage(s) of parasitic worm or nematode or filarial worm development. In such cases, “selective” means that the agent exhibits greater efficacy against one developmental stage as compared with its efficacy against another developmental stage. In particular embodiments, selective agents are at least about 3, 10, 30, 100, 300, or 1000-fold more effective at killing one stage of parasitic worm or nematode or filarial worm versus another stage. In some embodiments, selective agents exhibit no detectable efficacy against non-target developmental stages.

In a fourth aspect, microfilariae are administered to the immunocompromised rodents, to allow for the screening and evaluation of APIs that selectively target this earlier stage of the filarial parasites. Adult filarial nematodes live in a tissue or the circulatory system of vertebrates (the “definitive hosts”). These mature worms release microfilariae into the bloodstream of the vertebrate host. The microfilariae are then taken up by blood-feeding arthropod vectors (the “intermediate hosts”). In the intermediate host, the microfilariae develop into infective larvae (e.g., L3 D. immitis larvae), which may be transmitted to a new definitive host. The presence of microfilariae in the host bloodstream is referred to as “microfilaraemia.” Although the methods of the fourth aspect specify that microfilariae are administered rather than other stages of filarial worm, the type of immunocompromised rodents used, the type of filarial worms used, the means of infection, the number of microfilariae used to infect the immunocompromised rodent, the stage of worms counted in infected tissue, the use of control immunocompromised rodents, the comparison of parasiticidal efficacy (filarial efficacy) to other compounds, and any other features of the methods can all be as described for the third aspect.

In other embodiments of any of the disclosed methods, any larval stage may be studied and/or targeted, including, but not limited to, Mf, L3, L4, L5 and young adult. In one embodiment, the L3 larval stage is used.

In a fifth aspect, provided are methods of treating parasitic worm infections (e.g., nematode parasite infections), comprising administering to a subject in need thereof an effective amount of a parasiticidal compound identified using the immunocompromised rodent model as described in the third or fourth aspect. Also provided are parasiticidal (e.g., filaricidal) compounds identified in the method of the third aspect for use in a method of treating a parasitic worm infection (e.g., nematode parasite infection) in a subject in need thereof. The subject can be, for example, a human, a non-human animal, a non-human mammal, a dog, a sheep, a goat, or a cow. In one example, the parasitic worm is a nematode. In one example, the nematode is a filarial worm, and the method is for treating filariasis (e.g., dirofilariasis, lymphatic filariasis, or onchocerciasis). For example, the filarial worm can be D. immitis, and the subject can be a dog. In another example, the nematode can be a gastrointestinal parasite. As another example, the nematode can be Strongyloides stercoralis (threadworm), the subject can be a human or a dog, and the method can be for treating strongyloidiasis. As another example, the nematode can be Haemonchus contortus, the subject can be a ruminant, and the method can be for treating haemonchosis. A ruminant is a mammal that chews the cud regurgitated from its rumen, such as a sheep, a goat, or a cow.

Other objects, features and aspects of the present invention are disclosed in, or are obvious from, the following Detailed Description. It is to be understood by one of ordinary skill in the art that the present discussion is a description of exemplary embodiments only and is not intended as limiting the broader aspects of the present invention, which broader aspects are embodied in the exemplary construction. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents. The contents of all references, published patents, and patents cited throughout the present application are hereby incorporated by reference in their entirety.

### Definitions

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

By “animal” it is intended mammals, birds, and the like. Animal or host includes mammals and human. The animal may be selected from the group consisting of, but not limited to: equine, canine, feline, ovine, bovine, swine, caprine, avian, primate, and fish. The term “animal” also includes an individual animal in all stages of development, including embryonic and fetal stages.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

The term “filariasis” as used herein refers to helminth infections that are caused by filarial nematodes. An infection is the colonization of a host organism by parasite species. Infections with human filarial nematodes can cause lymphatic filariasis or onchocerciasis. The term “lymphatic filariasis” refers to an infection with the nematodes Wuchereria bancrofti, Brugia malayi or Brugia timori. The term “onchocerciasis” refers to an infection with the nematode Onchocerca volvulus. Lymphatic filariasis may cause hydrocoele, lymphoedema, and elephantiasis. Onchocerciasis may cause skin inflammation and blindness, so called River Blindness. In dogs, an infection with nematode species called Dirofilaria immitis (D. immitis) and Dirofilaria repens (D. repens) causes dirofilariasis. The former nematode species cause HW disease, while the latter causes subcutaneous dirofilariasis. As such, when the term “heartworm” (HW) is used to refer to the nematode, it is referring to D. immitis, not D. repens.

As used herein, the term “prophylactic treatment” refers to either preventing or inhibiting the development of a clinical condition or disorder or delaying the onset of a pre-clinically evident stage of a clinical condition or disorder. The term “prophylactic treatment” according to the present invention is to be understood as meaning that the compositions according to the present invention can be applied before symptoms of the infection are manifest. The term “prophylactic treatment” is to be understood as meaning a medical treatment. The compounds identified using the immunocompromised rodent model, according to the present invention can, for example, be used in a prophylactic treatment.

The term “therapeutically effective amount” is used herein to mean an amount or dose sufficient to cause an improvement in a clinically significant condition in the subject.

As used herein, “IVM” is ivermectin; “EMO” is emodepside; “8800” is depsipeptide 0798800; “4838” is depsipeptide 0804838; and “8914” is luxabendazole; and “6557” is a DI3 analog. “3615,” “8800,” “4838,” “7823,” “5010,” and “2677” are depsipeptide analogs described in US 2017/0022253 A1 and US 2017/0233438 A1, each of which is herein incorporated by reference in its entirety for all purposes. Structures for these compounds are provided below.

Unless otherwise defined, the technical and scientific terms used herein have the same meaning as commonly understood by one of ordinary skill in the art to which the present invention belongs.

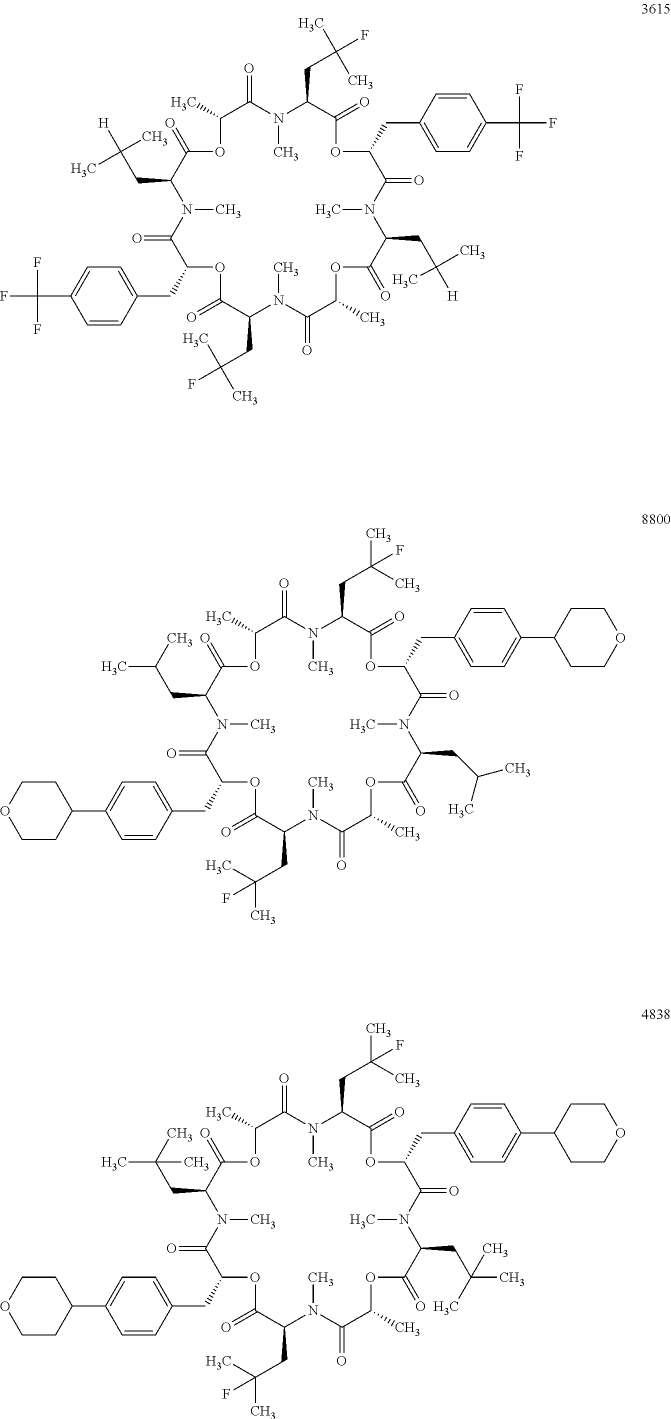

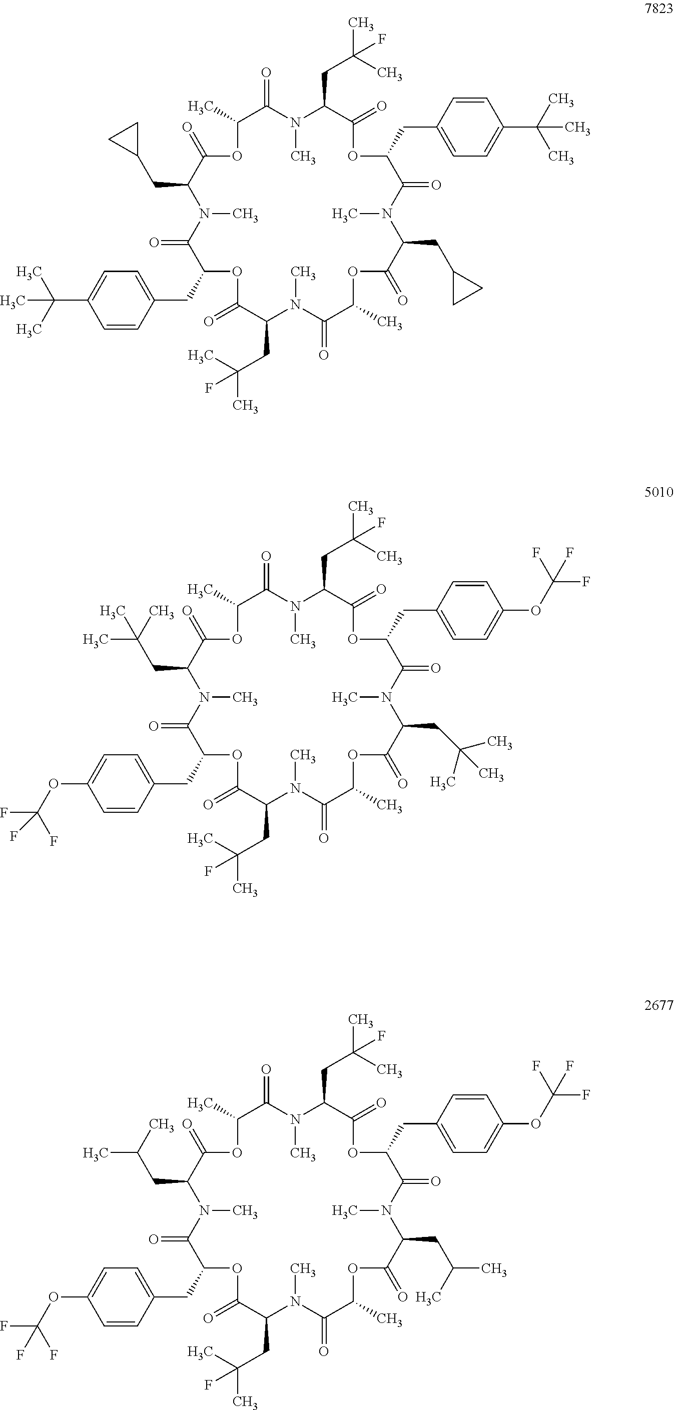

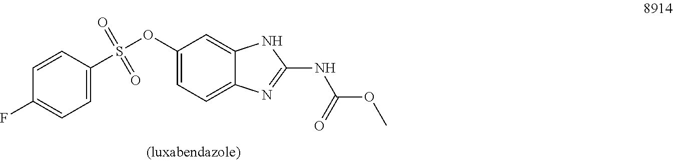 [SMILES: COC(=O)Nc1nc2ccc(OS(=O)(=O)c3ccc(F)cc3)cc2[nH]1]

### LISTING OF EMBODIMENTS

The subject matter disclosed herein includes, but is not limited to, the following embodiments:

1. A method of determining the effectiveness of a treatment for preventing filarial worm infection in an animal comprising: a. infecting an immunocompromised rodent with a specific number filarial worm larvae; b. treating the infected rodent with a compound or combination of compounds to be tested; and c. necropsying the rodent and determining the number of filarial worms present in the rodent tissue.

2. The method of embodiment 1, wherein the immunocompromised rodent is a mouse.

3. The method of embodiment 1 or 2, wherein the worm is Dirofilaria immitis (Heartworm; HW).

4. The method of any one of the preceding embodiments, further comprising the step of comparing the parasiticidal efficacy of the test compound(s) against the parasiticidal efficacy of either or both a macrocyclic lactone (ML) parasiticidal compound or a depsipeptide parasiticidal compound.

5. The method of embodiment 4, wherein the ML compound is selected from avermectin, ivermectin, doramectin, abamectin, milbemycin and moxidectin, 6. The method of embodiment 4 or 5, wherein the depsipeptide parasiticidal compound is emodepside.

7. Use of an immunocompromised rodent for modeling complete infection of a parasitic worm, outside of the worm's definitive host, wherein the rodent is characterized by absence of mature T- and B-cells and lack of functional NK cells.

8. Use of the rodent of embodiment 7, wherein the immunocompromised rodent is further characterized by a deficiency in cytokine signaling and multiple defects in innate immunity.

9. Use of the rodent of embodiment 7 or 8, wherein the immunocompromised rodent is further characterized by absence of production of detectable serum immunoglobulin.

10. Use of the rodent of any one of embodiments 7 to 9, wherein the Prkdc-gene of the rodent comprises a severe combined immunodeficiency (scid) mutation (Prkdc^{scid}).

11. Use of the rodent of any one of embodiment 7 to 10, wherein the rodent is a mouse, and wherein the mouse is a NOD scid IL2 receptor gamma chain knockout mouse (NSG) (NOD-SCID-IL2Ry −/−).

12. Use of the rodent of any one of embodiment 7 to 11, wherein the rodent has been genetically engineered to disrupt or delete genes required for wild type immune function.

13. A method of drug screening wherein the method comprises administering parasiticidal compounds or biologics to an immunocompromised rodent as defined in any one of embodiments 7 to 12.

14. A method of characterizing changes in sensitivity to a filaricidal agent during filarial worm infection by using a rodent as defined in any one of embodiments 7 to 12.

15. A method for treating filariasis, the method comprising administering to a subject in need thereof an effective amount of a filaricidal compound identified using the model immunocompromised rodent as defined in any one of embodiments 7 to 12.

16. The method of embodiment 15, wherein the filariasis is selected from filariasis and onchocerciasis.

17. The method of embodiment 15, wherein the filariasis is dirofilariasis.

18. The method of embodiment 15, wherein the subject is a human.

19. The method of embodiment 15, wherein the subject is a non-human animal.

20. The method of embodiment 15, wherein the subject is a dog.

21. The method of embodiment 15, wherein the subject is a dog and the filariasis is dirofilariasis.

22. The method of embodiment 15, wherein the subject is an animal and the filariasis is dirofilariasis.

23. The method of embodiment 15, wherein the subject is a human and the filariasis comprises lymphatic filariasis or onchocerciasis

The subject matter disclosed herein also includes, but is not limited to, the following embodiments:

24. A method of determining the effectiveness of a treatment for preventing filarial worm infection in an animal, preferably in a mammal, more preferably in a rodent, in particular in a rodent model, comprising: (a) infecting an immunocompromised rodent with a specific number of filarial worm larvae; (b) treating the infected rodent with a compound or combination of compounds to be tested; and (c) necropsying the rodent and determining the number of filarial worms present in the rodent tissue.

25. A method of determining the number of filarial worms in one or more isolated tissue sample(s) of an immunocompromised rodent, preferably necropsied immunocompromised rodent, infected with a specific number of filarial worm larvae and treated with a compound or combination of compounds to be tested, wherein such method comprises determining the number of filarial worms presented in such isolated tissue sample(s).

26. The method of embodiment 24 or 25, wherein the filarial worm larvae and/or filarial worms are selected from the group consisting of microfilariae, L3 larvae, L4 larvae, L5 larvae, young adult/mature worms, and/or adult/mature worms.

27. The method of any one of embodiments 24 to 26, wherein: (i) the immunocompromised rodent has been genetically engineered to disrupt or delete genes required for wild type immune function; and/or (ii) wherein the immunocompromised rodent is characterized by absence of mature T- and B-cells and lack of functional NK cells; and/or (iii) wherein the immunocompromised rodent is characterized by a deficiency in cytokine signaling and multiple defects in innate immunity; and/or (iv) wherein the immunocompromised rodent is characterized by absence of production of detectable serum immunoglobulin; and/or (v) wherein the Prkdc-gene of the immunocompromised rodent comprises a severe combined immunodeficiency (scid) mutation (Prkdc^{scid}); and/or (vi) wherein the rodent is a mouse, preferably an NOD scid IL2 receptor gamma chain knockout mouse (NSG) (NOD-SCID-IL2Ry −/−) or an NRG (NOD-Rag1^{null}IL2rg^{null})-immunodeficient mouse.

28. The method of any one of embodiments 24 to 27, wherein the filarial worm is Dirofilaria immitis (Heartworm; HW; Di) or Strongyloides stercoralis.

29. The method of any one embodiments 24 to 28, further comprising the step of comparing the parasiticidal efficacy of the test compound(s) against the parasiticidal efficacy of either or both a macrocyclic lactone (ML) parasiticidal compound and/or a depsipeptide parasiticidal compound.

30. The method of embodiment 29, wherein the ML compound is selected from avermectin, ivermectin, doramectin, abamectin, milbemycin and moxidectin.

31. The method of embodiment 29 or embodiment 30, wherein the depsipeptide parasiticidal compound is emodepside.

32. Use of an immunocompromised rodent for modeling complete infection of a parasitic worm, outside of the worm's definitive host, wherein: (i) the immunocompromised rodent has been genetically engineered to disrupt or delete genes required for wild type immune function; and/or (ii) wherein the immunocompromised rodent is characterized by absence of mature T- and B-cells and lack of functional NK cells; and/or (iii) wherein the immunocompromised rodent is characterized by a deficiency in cytokine signaling and multiple defects in innate immunity; and/or (iv) wherein the immunocompromised rodent is characterized by absence of production of detectable serum immunoglobulin; and/or (v) wherein the Prkdc-gene of the immunocompromised rodent comprises a severe combined immunodeficiency (scid) mutation (Prkdc^{scid}); and/or (vi) wherein the rodent is a mouse, preferably an NOD scid IL2 receptor gamma chain knockout mouse (NSG) (NOD-SCID-IL2Ry −/−) or an NRG (NOD-Rag1^{null}IL2rg^{null})-immunodeficient mouse.

33. Use of an immunocompromised rodent for a method of drug screening wherein the method comprises administering parasiticidal compounds or biologics to such immunocompromised rodent; or for a method of evaluating the efficacy of known or suspected parasiticidal, including filaricidal and microfilaricidal, compounds, wherein optionally or preferably in such methods the methods for treatment of the animal body by therapy and diagnostic methods practiced on the animal body are excluded, and wherein such immunocompromised rodent is the rodent according to embodiment 32.

34. Use of an immunocompromised rodent for a method of characterizing changes in sensitivity to a filaricidal agent during filarial worm infection, wherein such immunocompromised rodent is the rodent according to embodiment 32.

35. One or more filaricidal compound(s) identified in a method as described in any one of embodiments 24 to 31, preferably selected from the group consisting of emodepside, ivermectin, luxabendazole (8914), depsipeptide 0804838 (4838), depsipeptide 0798800 (8800), and/or DI₃ analog (6557) for use in a method of treating filariasis in a subject in need thereof, wherein preferably the filariasis is selected from filariasis and onchocerciasis, wherein more preferably the filariasis is dirofilariasis.

36. The one or more filaricidal compound(s) for use as described in embodiment 35, wherein the subject is a human or a non-human animal, preferably a dog.

37. The one or more filaricidal compound(s) for use as described in any one of embodiments 35 to 36, wherein the subject is an animal, preferably a dog, and the filariasis is dirofilariasis; or wherein the subject is a human and the filariasis comprises lymphatic filariasis or onchocerciasis.

38. An immunocompromised rodent, in particular the immunocompromised rodent according to embodiment 27 or embodiment 32, for use in a method according to any one of embodiments 24 to 26, 28 to 31 and 33 to 34.

The subject matter disclosed herein also includes, but is not limited to, the following embodiments:

39. A method of determining the effectiveness of a compound or combination of compounds for treating or preventing a parasitic worm infection comprising: (a) infecting an immunocompromised rodent with a specific number of parasitic worm larvae; (b) treating the infected rodent with the compound or combination of compounds to be tested; and (c) necropsying the rodent and determining the number of parasitic worms, optionally adult parasitic worms, present in a rodent tissue (e.g., an infected rodent tissue).

40. The method of embodiment 39, further comprising: (d) infecting a control immunocompromised rodent with a specific number parasitic worm larvae; (e) necropsying the control immunocompromised rodent and determining the number of worms, optionally adult parasitic worms, present in a rodent tissue (an infected rodent tissue); and (f) comparing the number of worms in the rodent tissue in step (c) with the number of worms in the rodent tissue in step (e).

41. A method of determining the number of parasitic worms in one or more isolated tissue sample(s) of an immunocompromised rodent, preferably necropsied immunocompromised rodent, infected with a specific number of parasitic worm larvae and treated with a compound or combination of compounds to be tested, wherein the method comprises determining the number of parasitic worms, optionally adult parasitic worms, present in the isolated tissue sample(s).

42. The method of embodiment 41, further comprising determining the number of parasitic worms in one or more isolated tissue sample(s) of an untreated immunocompromised rodent, preferably necropsied immunocompromised rodent, infected with a specific number of parasitic worm larvae, wherein the method comprises determining the number of parasitic worms, optionally adult parasitic worms, present in the isolated tissue sample(s) from the untreated immunocompromised rodent, and comparing the number of parasitic worms present in the isolated tissue sample(s) from the untreated immunocompromised rodent with the number of parasitic worms present in the isolated tissue sample(s) from the treated immunocompromised rodent.

43. The method of any one of embodiments 39 to 42, wherein the parasitic worm is a nematode.

44. The method of embodiment 43, wherein the parasitic worm is a filarial worm, optionally wherein the filarial worm is Dirofilaria immitis.

45. The method of embodiment 43, wherein the parasitic worm is a gastrointestinal parasitic worm, optionally wherein the gastrointestinal parasitic worm is Strongyloides stercoralis or Haemonchus contortus.

46. The method of any one of embodiments 39 to 45, wherein the parasitic worm larvae are selected from the group consisting of microfilariae, L3 larvae, and L4 larvae.

47. The method of any one of embodiments 39 to 46, wherein: (i) the immunocompromised rodent has been genetically engineered to disrupt or delete genes required for wild type immune function; and/or (ii) the immunocompromised rodent is characterized by absence of mature T and B cells and lack of functional NK cells; and/or (iii) the immunocompromised rodent is characterized by a deficiency in cytokine signaling and multiple defects in innate immunity; and/or (iv) the immunocompromised rodent is characterized by absence of production of detectable serum immunoglobulin; and/or (v) the Prkdc gene of the immunocompromised rodent comprises a severe combined immunodeficiency (scid) mutation (Prkdc^{scid}); and/or (vi) the rodent is a mouse, preferably an NOD scid IL2 receptor gamma chain knockout mouse (NSG) (NOD-SCID-IL2Ry −/−) or an NRG (NOD-Rag1^{null}IL2rg^{null}) immunodeficient mouse.

48. The method of any one of embodiments 39 to 47, wherein the immunocompromised rodent does not comprise engrafted immune cells from a different species.

49. The method of any one of embodiments 39 to 48, wherein the immunocompromised rodent comprises engrafted immune cells from a definitive host of the parasitic worm, optionally wherein the engrafted immune cells are canine immune cells.

50. The method of any one embodiments 39 to 49, further comprising comparing the parasiticidal efficacy of the compound or combination of compounds against the parasiticidal efficacy of either or both a macrocyclic lactone (ML) parasiticidal compound and/or a depsipeptide parasiticidal compound.

51. The method of embodiment 50, wherein the ML compound is selected from avermectin, ivermectin, doramectin, abamectin, milbemycin and moxidectin.

52. The method of embodiment 50 or 51, wherein the depsipeptide parasiticidal compound is emodepside.

53. The method of any one of embodiments 39 to 52, wherein the immunocompromised rodent is an NSG or an NRG mouse, and wherein the parasitic worm is Dirofilaria immitis.

54. The method of any one of embodiments 39 to 47 and 49 to 53, wherein the immunocompromised rodent is an NSG or an NRG mouse engrafted with canine immune cells, and wherein the parasitic worm is Dirofilaria immitis.

55. One or more filaricidal compound(s) identified by the method of any one of embodiments 39 to 54, preferably selected from the group consisting of emodepside, ivermectin, luxabendazole (8914), depsipeptide 0804838 (4838), depsipeptide 0798800 (8800), and/or DI₃ analog (6557), for use in a method of treating filariasis in a subject in need thereof, wherein preferably the filariasis is dirofilariasis.

56. The one or more filaricidal compound(s) for use as described in embodiment 55, wherein the subject is a dog and the filariasis is dirofilariasis.

57. One or more parasiticidal compound(s) identified by the method of any one of embodiments 39 to 54 for use in a method of treating a gastrointestinal parasite infection in a subject in need thereof, wherein preferably the gastrointestinal parasite infection is haemonchosis or strongyloidiasis.

58. The one or more parasiticidal compound(s) for use as described in embodiment 57, wherein the subject is a dog or human, and the gastrointestinal parasite infection is strongyloidiasis.

59. The one or more parasiticidal compound(s) for use as described in embodiment 57, wherein the subject is a ruminant, and the gastrointestinal parasite infection is haemonchosis.

60. An immunocompromised rodent infected with one or more adult parasitic worms.

61. The immunocompromised rodent of embodiment 60, wherein the parasitic worm is a nematode.

62. The immunocompromised rodent of embodiment 61, wherein the parasitic worm is a filarial worm, optionally wherein the filarial worm is Dirofilaria immitis.

63. The immunocompromised rodent of embodiment 61, wherein the parasitic worm is a gastrointestinal parasitic worm, optionally wherein the gastrointestinal parasitic worm is Strongyloides stercoralis or Haemonchus contortus.

64. The immunocompromised rodent of any one of embodiments 60 to 63, wherein: (i) the immunocompromised rodent has been genetically engineered to disrupt or delete genes required for wild type immune function; and/or (ii) the immunocompromised rodent is characterized by absence of mature T and B cells and lack of functional NK cells; and/or (iii) the immunocompromised rodent is characterized by a deficiency in cytokine signaling and multiple defects in innate immunity; and/or (iv) the immunocompromised rodent is characterized by absence of production of detectable serum immunoglobulin; and/or (v) the Prkdc gene of the immunocompromised rodent comprises a severe combined immunodeficiency (scid) mutation (Prkdc^{scid}); and/or (vi) the rodent is a mouse, preferably an NOD scid IL2 receptor gamma chain knockout mouse (NSG) (NOD-SCID-IL2Ry −/−) or an NRG (NOD-Rag1^{null}IL2rg^{null}) immunodeficient mouse.

65. The immunocompromised rodent of any one of embodiments 60 to 64, wherein the rodent is a mouse.

66. The immunocompromised rodent of any one of embodiments 60 to 65, wherein the immunocompromised rodent is an NSG mouse or an NRG mouse, and wherein the parasitic worm is Dirofilaria immitis.

67. The immunocompromised rodent of any one of embodiments 60 to 66, wherein the immunocompromised rodent does not comprise engrafted immune cells from a different species.

68. The immunocompromised rodent of any one of embodiments 60 to 66, wherein the immunocompromised rodent comprises engrafted immune cells from a definitive host of the parasitic worm, optionally wherein the engrafted immune cells are canine immune cells.

69. The immunocompromised rodent of any one of embodiments 60 to 66 and 68, wherein the immunocompromised rodent is an NSG mouse or an NRG mouse comprising engrafted canine immune cells, and wherein the parasitic worm is Dirofilaria immitis.

70. The immunocompromised rodent of any one of embodiments 60 to 69 for use in a method of determining the effectiveness of a compound for treating or preventing a parasitic worm infection, wherein optionally or preferably in such methods the methods for treatment of the animal body by therapy and diagnostic methods practiced on the animal body are excluded.

71. The immunocompromised rodent of any one of embodiments 60 to 69 for use in a method of modeling complete infection of a parasitic worm outside of the worm's definitive host.

72. The immunocompromised rodent of any one of embodiments 60 to 69 for use in a method of drug screening wherein the method comprises administering parasiticidal compounds or biologics to the immunocompromised rodent; or for a method of evaluating the efficacy of known or suspected parasiticidal, including filaricidal and microfilaricidal, compounds, wherein optionally or preferably in such methods the methods for treatment of the animal body by therapy and diagnostic methods practiced on the animal body are excluded.

73. The immunocompromised rodent of any one of embodiments 60 to 69 for use in a method of characterizing changes in sensitivity to a parasiticidal or filaricidal agent during parasitic or filarial worm infection.

74. Use of an immunocompromised rodent for modeling complete infection of a parasitic worm outside of the worm's definitive host, wherein: (i) the immunocompromised rodent has been genetically engineered to disrupt or delete genes required for wild type immune function; and/or (ii) the immunocompromised rodent is characterized by absence of mature T- and B-cells and lack of functional NK cells; and/or (iii) the immunocompromised rodent is characterized by a deficiency in cytokine signaling and multiple defects in innate immunity; and/or (iv) the immunocompromised rodent is characterized by absence of production of detectable serum immunoglobulin; and/or (v) the Prkdc gene of the immunocompromised rodent comprises a severe combined immunodeficiency (scid) mutation (Prkdc^{scid}); and/or (vi) the rodent is a mouse, preferably an NOD scid IL2 receptor gamma chain knockout mouse (NSG) (NOD-SCID-IL2Ry −/−) or an NRG (NOD-Rag1^{null}IL2rg^{null}) immunodeficient mouse.

75. Use of an immunocompromised rodent for a method of drug screening, wherein the method comprises administering parasiticidal compounds or biologics to the immunocompromised rodent; or for a method of evaluating the efficacy of known or suspected parasiticidal, including filaricidal and microfilaricidal, compounds, wherein optionally or preferably in such methods the methods for treatment of the animal body by therapy and diagnostic methods practiced on the animal body are excluded, and wherein the immunocompromised rodent is the rodent according to embodiment 74.

76. Use of an immunocompromised rodent for a method of characterizing changes in sensitivity to a filaricidal agent during filarial worm infection, wherein the immunocompromised rodent is the rodent according to embodiment 74.

77. An immunocompromised rodent for use in a method according to any one of embodiments 39 to 54 and 74 to 76.

The following examples are simply intended to further illustrate and explain the present invention. The examples, therefore, should not be regarded as limiting the scope of the invention or manner in which it may be practiced.

### EXAMPLES

For the following studies, D. immitis were obtained from FR3, the Merial insectary and Joe Prullage's group at MRC. As used herein, “FR3” refers the Filariasis Research Reagent Resource Center at the University of Georgia Vet School, which supplied Applicants with ML-susceptible 2005 Missouri strain Di. In general, worms were recovered from mosquitos and shipped overnight in media and antibiotics. Worms were washed and counted for use in mice. Mice were injected subcutaneously or via intraperitoneal injection. For the recovery of parasites, mice were exsanguinated for serum collection, skinned and the fascia scored, the viscera (pluck) removed, and the muscle fascia scored. All pieces were left to soak overnight room temperature in RPMI media supplemented with 10% FBS and Pen/strep antibiotics. The following morning, approximately 18 hours later, worms were counted and fixed in 95% ethanol/5% glycerol. Worms were mounted in glycerine jelly and then measured using cellSens software.

### Example 1—Development and Validation of an Intermediate In Vivo Model of Canine Heartworm Infection

Canine heartworm (HW), D. immitis, related to the human river-blindness parasite O. volvulus, is transmitted via a mosquito vector that delivers infective L3 larva to hosts. The parasite matures through several larval stages, migrating through host tissue to the pulmonary arteries and heart. Adult heartworms can grow to 20-30 cm in length. Adults produce circulating microfilariae that are transferred to the mosquito in a bloodmeal. Once internalized, the microfilaria mature to infective L3 larvae, completing the cycle.

However, prior to the present disclosure, there existed a significant challenge for translational biologists: Dirofilaria immitis (Di) larvae fail to develop in immunocompetent rodents (see Table 2). “Mouse #” refers to the identifier for a particular mouse. The numbers in the “Pluck,” “Upper Muscles,” “Lower Muscles,” “Skin,” “Total,” and “Average Recovery” columns refer to number of Di recovered. Timelines, cost, and risk were thus increased due to sole reliance upon canine studies.

TABLE 2
D. immitis larvae do not survive in C57/BL6 mice.

| C57/BL6 | | Upper | Lower | | | Average |
|---|---|---|---|---|---|---|
| Mouse # | Pluck | Muscles | Muscles | Skin | Total | Recovery |
| 1 | 0 | 0 | 0 | 0 | 0 | 0 |
| 2 | 0 | 0 | 0 | 0 | 0 | |
| 3 | 0 | 0 | 0 | 0 | 0 | |
| 4 | 0 | 0 | 0 | 0 | 0 | |

Surprisingly, Applicants found that immunodeficient NSG mice were susceptible to D. immitis infection and complete development. Following subcutaneous (SQ) infection with 100 infective L3 stage larva, D. immitis can be recovered from NSG mouse tissues (Table 3), indicating migration through the murine host similar to that which occurs in canine infections.

TABLE 3
Recovery of D. immitis from NSG mice 6 weeks post SQ infection.

| NSG | | Upper | Lower | | | Average |
|---|---|---|---|---|---|---|
| Mouse # | Pluck | Muscles | Muscles | Skin | Total | Recovery |
| 1 | 0 | 10 | 10 | 13 | 33 | 25 |
| 2 | 0 | 9 | 1 | 5 | 15 | |
| 3 | 0 | 3 | 11 | 11 | 25 | |
| 4 | 0 | 12 | 8 | 8 | 28 | |

As disclosed herein, the mouse model of canine HW infection promises to accelerate discovery of new pharmaceutical treatments by reducing timelines (6 weeks in mouse, compared to 6 months in canines) and costs.

Similar results were obtained when D. immitis L3 larvae were introduced into NSG or C57/BL6 mice via the intraperitoneal (IP) route (Table 4).

TABLE 4
Recovery of D. immitis from NSG mice 6 weeks post IP infection.

| | | Mouse | | Upper | Lower | | | Average |
|---|---|---|---|---|---|---|---|---|
| Source | Sex | # | Pluck | Muscles | Muscles | Skin | Total | Recovery |
| NSG | M | 1 | 0 | 5 | 1 | 2 | 8 | 8 |
| | M | 2 | 0 | 3 | 3 | 2 | 8 | |
| C57/BL6 | M | 3 | 0 | 0 | 0 | 0 | 0 | 0 |
| | M | 4 | 0 | 0 | 0 | 0 | 0 | |
| | M | 5 | 0 | 0 | 0 | 0 | 0 | |

To determine the effect of varying the dose, NSG mice were injected SQ as described above, this time with 25, 50 or 100 D. immitis L3 larvae (Table 5). “Mouse #” refers to the identifier for a particular mouse. The numbers in the “Pluck,” “Upper Muscles,” “Lower Muscles,” “Skin,” “Total,” and “Average Recovery” columns refer to number of Di recovered. “% Recovery” refers to percent of the original 25, 50, or 100 Di recovered from the mouse. And while there was a statistically significant dose-dependent increase in average recovery, percent recovery did not change significantly across the tested doses.

TABLE 5
Recovery of D. immitis from NSG mice 6 weeks
post SQ infection with 25, 50, or 100 L3 larvae.

| | | Mouse | | Upper | Lower | | | Average | % |
|---|---|---|---|---|---|---|---|---|---|
| Dose | Sex | # | Pluck | Muscles | Muscles | Skin | Total | Recovery | Recovery |
| 25 | F | 9 | 0 | 1 | 0 | 4 | 5 | 4 | 16 |
| | M | 11 | 0 | 2 | 0 | 0 | 2 | | |
| | M | 12 | 0 | 5 | 0 | 2 | 7 | | |
| | F | 16 | 1 | 0 | 0 | 2 | 3 | | |
| | F | 17 | 0 | 0 | 1 | 2 | 3 | | |
| | F | 18 | 0 | 3 | 1 | 0 | 4 | | |
| 50 | F | 7 | 0 | 4 | 1 | 2 | 7 | 11 | 22 |
| | F | 8 | 1 | 2 | 7 | 4 | 14 | | |
| | F | 10 | 0 | 1 | 4 | 5 | 10 | | |
| | M | 13 | 0 | 6 | 0 | 3 | 9 | | |
| | M | 14 | 0 | 6 | 6 | 4 | 16 | | |
| | M | 15 | 0 | 2 | 2 | 7 | 11 | | |
| 100 | M | 1 | 0 | 2 | 5 | 10 | 17 | 21 | 21 |
| | M | 2 | 0 | 6 | 8 | 8 | 22 | | |
| | M | 3 | 0 | 3 | 10 | 9 | 22 | | |
| | M | 4 | 1 | 2 | 4 | 1 | 8 | | |
| | F | 5 | 1 | 14 | 6 | 11 | 32 | | |
| | F | 6 | 0 | 11 | 8 | 5 | 24 | | |

** ANOVA indicates each group is different from each other based on actual recovery numbers
***Same analysis on % survival shows no difference between groups

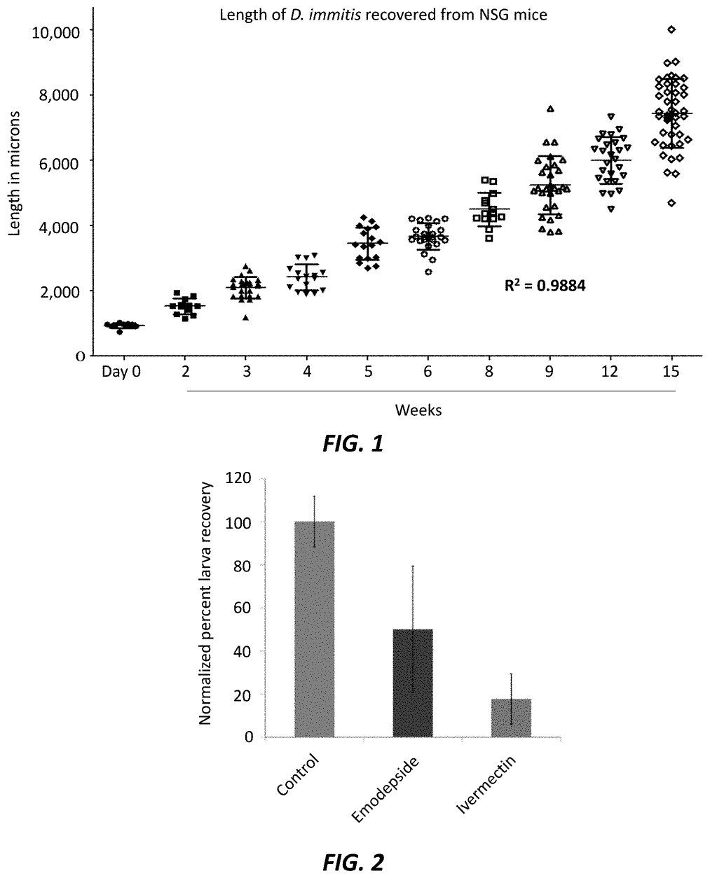

To test the linearity of the NSG mouse model of D. immitis infection, NSG mice were next infected with 100 L3 per mouse, using methods described above. Recovery and measurements were performed as above, and lengths were reported as a function of time post-infection (FIG. 1). As indicated, the model is significantly and surprisingly linear, with a calculated R² value of 0.9884.

To test whether HW could be eliminated from the NSG mouse model by pharmaceutical compounds, Applicants tested known filaricidal compounds, emodepside (EMO) and ivermectin (IVM). NSG mice were dosed orally with 1 mg/kg EMO or 3 mg/kg IVM, at Days 1, 15, and 30 post-infection. As indicated in FIG. 2, both compounds significantly reduced HW parasite load.

In view of these promising data, Applicants envision that the immunocompromised rodent model may be successfully applied to a wide range of filarial parasites to predict compound efficacy in animals, including canines. Applicants further envision that HW parasites in NSG mice may be physiologically similar to those from their normal, definitive canine hosts.

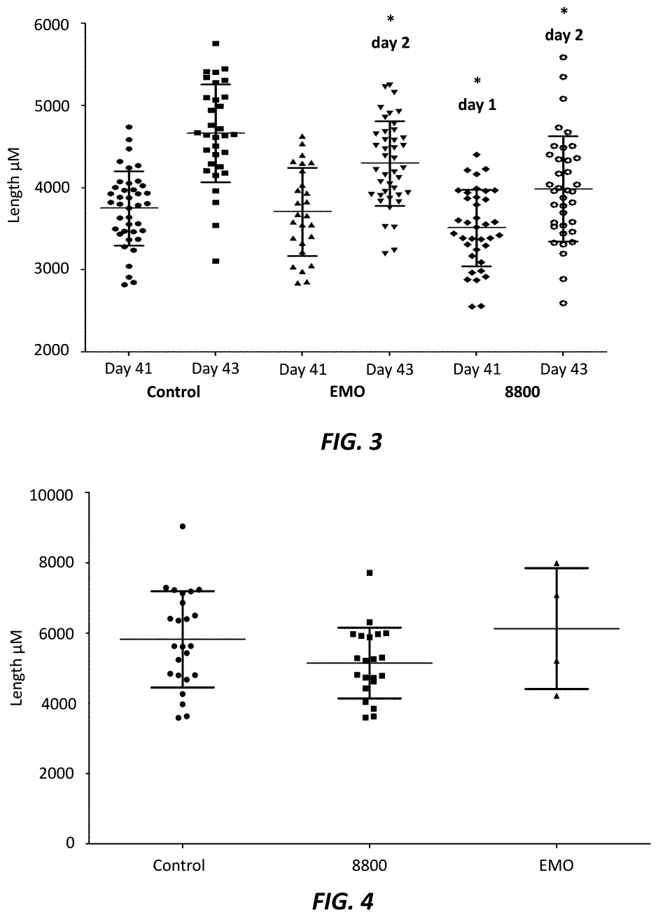

In another example, NSG mice were infected on Day 0 with 50 Di L3 larvae. The mice were then treated with 1 mg/kg EMO or 8800 on Day 30, and recovery was conducted on Days 41 to 43. FIG. 3 shows the length of Di recovered from the mice at Days 41 and 43, and Table 6 provides survival data, recovery data, and summary statistics. “#” refers to the mouse identifier for a particular mouse. “Survival” refers to the number of Di recovered from the mouse. “% Rec” refers to percent of the original 50 Di recovered from the mouse. “% Red” refers to percent reduction in the percent of Di recovered compared to control.

TABLE 6
Summary data for Di-infected NSG mice
treated with Control, EMO, or 8800.

| Control | | | EMO | | | 8800 | | |
|---|---|---|---|---|---|---|---|---|
| | | % | | | % | | | % |
| # | Survival | Rec | # | Survival | Rec | # | Survival | Rec |
| 1 | 18 | 36 | 13 | 15 | 30 | 29 | 14 | 28 |
| 3 | 18 | 36 | 14 | 13 | 26 | 30 | 26 | 52 |
| 5 | 21 | 42 | 17 | 12 | 24 | 33 | 12 | 24 |
| 9 | 20 | 40 | 18 | 15 | 30 | 34 | 18 | 36 |
| 10 | 23 | 46 | 19 | 16 | 32 | 35 | 12 | 24 |
| | | | 20 | 14 | 28 | 36 | 12 | 24 |
| Mean | 20 | 40 | Mean | 14 | 28 | Mean | 16 | 31 |
| SD | 2 | 4 | SD | 1 | 3 | SD | 6 | 11 |

| | | | |
|---|---|---|---|
| % | 30% | % | 23% |
| Red | | Red | |
| P | 0.019 | P | 0.069 |
| value | | value | |

In another example, NSG mice were infected at Day 0 with 50 Di L3 larvae. The mice were then treated with Control; 1 mg/kg EMO; or 8800 on Days 30 and 60. FIG. 4 shows the lengths of Di recovered at Days 83 and 84, and Table 7 presents the survival data, recovery data, and summary statistics.

TABLE 7
Summary data for Di-infected NSG mice
treated with Control, EMO, or 8800.

| Control | | | EMO | | | 8800 | | |
|---|---|---|---|---|---|---|---|---|
| | | % | | | % | | | % |
| # | Survival | Rec | # | Survival | Rec | # | Survival | Rec |
| 6 | 11 | 22 | 15 | 1 | 2 | 31 | 6 | 12 |
| 7 | 5 | 10 | 16 | 0 | 0 | 32 | 3 | 6 |
| 8 | 7 | 14 | 21 | 1 | 2 | 37 | 7 | 14 |
| 11 | 5 | 10 | 23 | 0 | 0 | 38 | 2 | 4 |
| 12 | 6 | 12 | 25 | 3 | 6 | 41 | 2 | 4 |
| | | | 26 | 1 | 2 | 42 | 5 | 10 |
| | | | 27 | 0 | 0 | | | |
| | | | 28 | 3 | 6 | | | |
| Mean | 7 | 14 | Mean | 1 | 2 | Mean | 4 | 8 |
| SD | 2 | 5 | SD | 1 | 2 | SD | 2 | 4 |
| | | | % | 86% | | % | 43% | |
| | | | Red | | | Red | | |
| | | | P | 0.000 | | P | 0.021 | |
| | | | value | | | value | | |

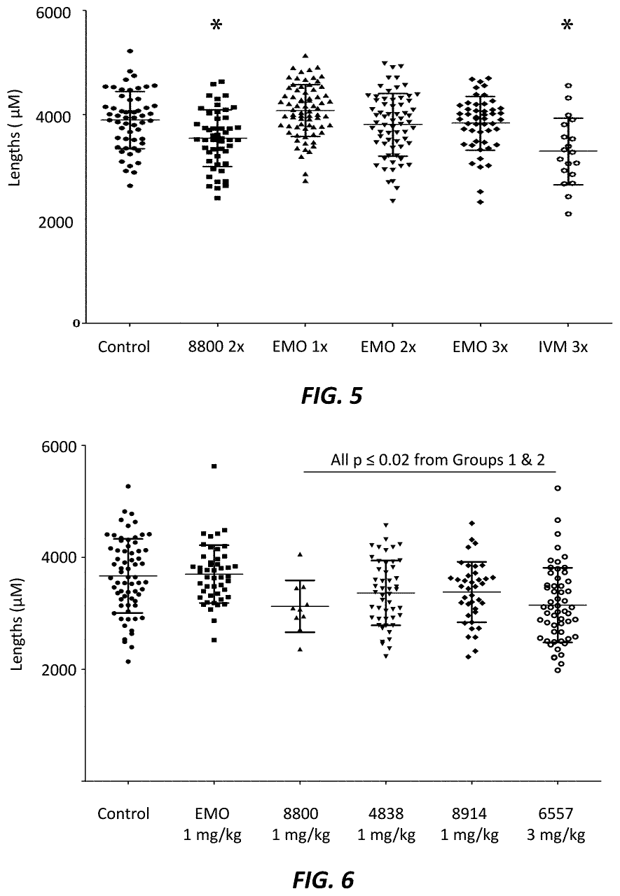

In another example, NSG mice were infected with 50 Di L3 per mouse, then treated according to the following: Control; 2× 8800 (1 mg/kg) at Days 1 and 30; 1× EMO (1 mg/kg) at Day 30; 2× EMO (1 mg/kg) at Days 1 and 30; 3× EMO (1 mg/kg) at Days 1, 15, and 30; or 3× IVM (3 mg/kg) at Days 1, 15, and 30. FIG. 5 is a graph showing the length of Di recovered at Days 41 to 43, and Table 8 presents the survival data, recovery data, and summary statistics.

TABLE 8
Summary data for Di-infected NSG mice treated with Control, 8800, EMO, or IVM.

| Control | | | 2 × 8800 | | | 1 × EMO | | | 2 × EMO | | | 3 × EMO | | | 3 × IVM | | |
|---|---|---|---|---|---|---|---|---|---|---|---|---|---|---|---|---|---|
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 16 | 32 | 7 | Died | | 14 | 21 | 42 | 21 | 13 | 26 | 28 | 14 | 28 | 35 | 6 | 12 |
| | | | | Jan. 14, 2016 | | | | | | | | | | | | | |
| 2 | 16 | 32 | 8 | 16 | 32 | 15 | 22 | 44 | 22 | 11 | 22 | 29 | 10 | 20 | 36 | 5 | 10 |
| 3 | 16 | 32 | 9 | 11 | 22 | 16 | 18 | 36 | 23 | 14 | 28 | 30 | 14 | 28 | 37 | 1 | 2 |
| 4 | 15 | 30 | 10 | 8 | 16 | 17 | 14 | 28 | 24 | 13 | 26 | 31 | 7 | 14 | 38 | 3 | 6 |
| 5 | 16 | 32 | 11 | 6 | 12 | 18 | 14 | 28 | 25 | 13 | 26 | 32 | 11 | 22 | 39 | 4 | 8 |
| 6 | 22 | 44 | 12 | 13 | 26 | 19 | 23 | 46 | 26 | 15 | 30 | 33 | 2 | 4 | 40 | 2 | 4 |
| | | | 13 | 10 | 20 | 20 | 23 | 46 | 27 | 21 | 42 | 34 | 3 | 6 | 41 | 1 | 2 |
| Mean | 17 | 34 | Mean | 11 | 21 | Mean | 19 | 39 | Mean | 14 | 29 | Mean | 9 | 17 | Mean | 3 | 6 |
| SD | 3 | 5 | SD | 4 | 7 | SD | 4 | 8 | SD | 3 | 6 | SD | 5 | 10 | SD | 2 | 4 |

| | | | | | | | | | |
|---|---|---|---|---|---|---|---|---|---|
| % | 38% | % | 0% | % | 15% | % | 50% | % | 82% |
| Red | | Red | | Red | | Red | | Red | |
| P | 0.005 | P | 0.218 | P | 0.201 | P | 0.000 | P | 0.000 |
| value | | value | | value | | value | | value | |

In another example, NSG mice were infected with 50 Di L3 per mouse, then treated according to the following: EMO (1 mg/kg); 8800 (1 mg/kg); 4838 (1 mg/kg); 8914 (1 mg/kg); and 6557 (3 mg/kg). FIG. 6 is a graph showing the lengths of Di recovered at Days 41 to 43; and, Table 9 presents the survival data, recovery data, and summary statistics.

TABLE 9
Summary data for Di-infected NSG mice treated with Control, EMO, 8800, 4838, 8914, or 6557.

| Control | | | EMO | | | 8800 | | | 4838 | | | 8914 | | | 6557 | | |
|---|---|---|---|---|---|---|---|---|---|---|---|---|---|---|---|---|---|
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 12 | 24 | 8 | 10 | 20 | 14 | 8 | 16 | 20 | 7 | 14 | 26 | 16 | 32 | 32 | 21 | 42 |
| 2 | 13 | 26 | 9 | 8 | 16 | 15 | 0 | 0 | 21 | 16 | 32 | 27 | 14 | 28 | 33 | 13 | 26 |
| 3 | 19 | 38 | 10 | 11 | 22 | 16 | 4 | 8 | 22 | 15 | 30 | 41 | 24 | 48 | 34 | 13 | 26 |
| 4 | 17 | 34 | 11 | 13 | 26 | 17 | 4 | 8 | 23 | 13 | 26 | 29 | 12 | 24 | 35 | Died 3/18 | |
| 5 | 19 | 38 | 12 | 12 | 24 | 18 | 5 | 10 | 24 | 15 | 30 | 30 | Died 3/14 | | 36 | 19 | 38 |
| 6 | 20 | 40 | 13 | 12 | 24 | 19 | 4 | 8 | 25 | 6 | 12 | 31 | Died 2/19 | | 37 | 27 | 54 |
| 7 | 14 | 28 | | | | 39 | 8 | 16 | 40 | 14 | 28 | 38 | Died 3/14 | | 42 | Died 3/3 | |
| | | | | | | | | | | | | 28 | Died 2/19 | | | | |
| Mean | 16 | 33 | Mean | 11 | 22 | Mean | 5 | 9 | Mean | 12 | 25 | Mean | 17 | 33 | Mean | 19 | 37 |
| SD | 3 | 7 | SD | 2 | 4 | SD | 3 | 6 | SD | 4 | 8 | SD | 5 | 11 | SD | 6 | 12 |

| | | | | | | | | | |
|---|---|---|---|---|---|---|---|---|---|
| % | 33% | % | 73% | % | 24% | % | 0% | % | 0% |
| Red | | Red | | Red | | Red | | Red | |
| P | 0.020 | P | 0.000 | P | 0.062 | P | 0.930 | P | 0.314 |
| value | | value | | value | | value | | value | |

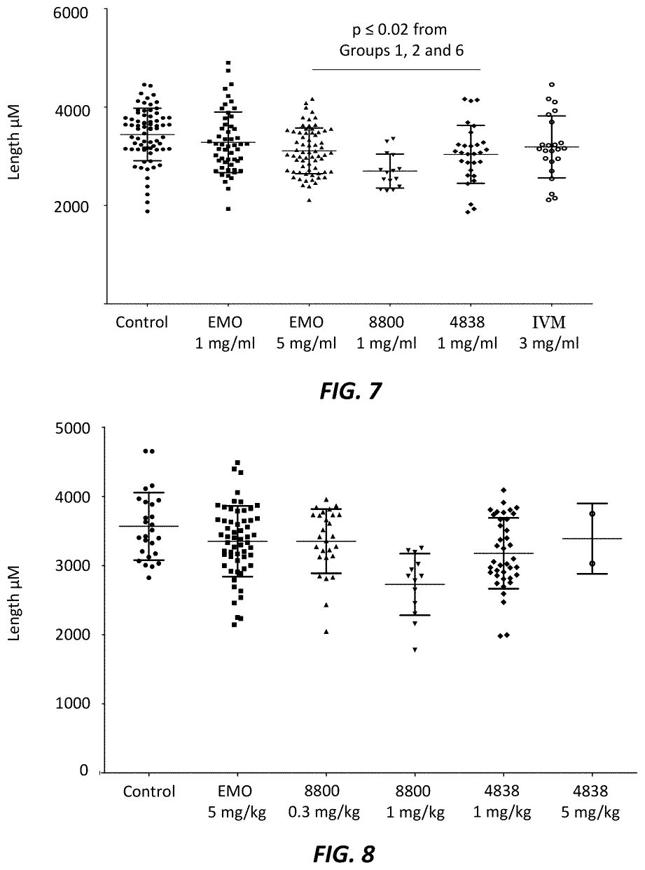

In another example, NSG mice were infected with 50 Di L3 Merial worms (not FR3 worms) per mouse, then treated on Days 1, 15, and 30 according to the following: EMO (1 mg/kg); EMO (5 mg/kg); 8800 (1 mg/kg); 4838 (1 mg/kg); or IVM (3 mg/kg). FIG. 7 is a graph showing the lengths of Di recovered at Days 41 to 43, and Table 10 presents the survival data, recovery data, and summary statistics.

TABLE 10
Summary data for Di-infected NSG mice treated with Control, EMO low dose, EMO high dose, 8800, 4838, or IVM.

| Control | | | EMO low dose | | | EMO High dose | | | 8800 | | | 4838 | | | IVM | | |
|---|---|---|---|---|---|---|---|---|---|---|---|---|---|---|---|---|---|
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 17 | 34 | 7 | 12 | 24 | 15 | 18 | 36 | 23 | 8 | 16 | 31 | 8 | 16 | 39 | 16 | 32 |
| 2 | 14 | 28 | 8 | 16 | 32 | 16 | 24 | 48 | 24 | 0 | 0 | 32 | 18 | 36 | 40 | 2 | 4 |
| 3 | 17 | 34 | 9 | 11 | 22 | 17 | 24 | 48 | 25 | 6 | 12 | 33 | 10 | 20 | 41 | 6 | 12 |
| 4 | 17 | 34 | 10 | 7 | 14 | 18 | 26 | 52 | 26 | 0 | 0 | 34 | 10 | 20 | 42 | 8 | 16 |
| 5 | 25 | 50 | 11 | 13 | 26 | 19 | 34 | 68 | 27 | 6 | 12 | 35 | 6 | 12 | 43 | 6 | 12 |
| 6 | 18 | 36 | 12 | 12 | 24 | 20 | 24 | 48 | 28 | 4 | 8 | 36 | 12 | 24 | 44 | 8 | 16 |
| 47 | 11 | 22 | 13 | 8 | 16 | 21 | 28 | 56 | 29 | 6 | 12 | 37 | 10 | 20 | 45 | 12 | 24 |
| | | | 14 | 10 | 20 | 22 | 24 | 48 | 30 | 16 | 32 | 38 | 8 | 16 | 46 | 14 | 28 |
| Mean | 17 | 34 | Mean | 11 | 22 | Mean | 25 | 51 | Mean | 6 | 12 | Mean | 10 | 21 | Mean | 9 | 18 |
| SD | 4 | 9 | SD | 3 | 6 | SD | 5 | 9 | SD | 5 | 10 | SD | 4 | 7 | SD | 5 | 9 |

| | | | | | | | | | |
|---|---|---|---|---|---|---|---|---|---|
| % | 35% | % | 0% | % | 65% | % | 38% | % | 47% |
| Red | | Red | | Red | | Red | | Red | |
| P | 0.010 | P | 0.001 | P | 0.000 | P | 0.004 | P | 0.000 |
| value | | value | | value | | value | | value | |

In another example, NSG mice were infected with 50 Di L3 per mouse, then treated on Days 1, 15, and 30 according to the following: EMO (5 mg/kg); 8800 (0.3 mg/kg); 8800 (1 mg/kg); 4838 (1 mg/kg); or 4838 (5 mg/kg). FIG. 8 is a graph showing the lengths of Di recovered at Days 41 to 42, and Table 11 presents the survival data, recovery data, and summary statistics.

TABLE 11
Summary data for Di-infected NSG mice treated with Control, EMO, 8800, or 4838.

| Control | | | EMO 5 mg/kg | | | 8800 0.3 mg/kg | | | 8800 1 mg/kg | | | 4838 1 mg/kg | | | 4838 5 mg/kg | | |
|---|---|---|---|---|---|---|---|---|---|---|---|---|---|---|---|---|---|
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 13 | 26 | 6 | 11 | 22 | 11 | 8 | 16 | 16 | 4 | 8 | 21 | 15 | 30 | 26 | 2 | 4 |
| 2 | 9 | 18 | 7 | 17 | 34 | 12 | 18 | 36 | 17 | 2 | 4 | 22 | 14 | 28 | 27 | 1 | 2 |
| 3 | 14 | 28 | 8 | 7 | 14 | 13 | 18 | 36 | 18 | 3 | 6 | 23 | 9 | 18 | 28 | 0 | 0 |
| 4 | 13 | 26 | 9 | 11 | 22 | 14 | 8 | 16 | 19 | 2 | 4 | 24 | 10 | 20 | 29 | 2 | 4 |
| 5 | 11 | 22 | 10 | 14 | 28 | 15 | 18 | 36 | 20 | 1 | 2 | 25 | 6 | 12 | 30 | 1 | 2 |
| 31 | 13 | 26 | 32 | 18 | 36 | 33 | 7 | 14 | 34 | 8 | 16 | 35 | 4 | 8 | 36 | 1 | 2 |
| Mean | 12 | 24 | Mean | 13 | 26 | Mean | 13 | 26 | Mean | 3 | 7 | Mean | 10 | 19 | Mean | 1 | 2 |
| SD | 2 | 4 | SD | 4 | 8 | SD | 6 | 11 | SD | 3 | 5 | SD | 4 | 9 | SD | 1 | 2 |

| | | | | | | | | | |
|---|---|---|---|---|---|---|---|---|---|
| % | 0% | % | 0% | % | 71% | % | 21% | % | 92% |
| Red | | Red | | Red | | Red | | Red | |
| P | 0.692 | P | 0.751 | P | 0.000 | P | 0.240 | P | 0.000 |
| value | | value | | value | | value | | value | |

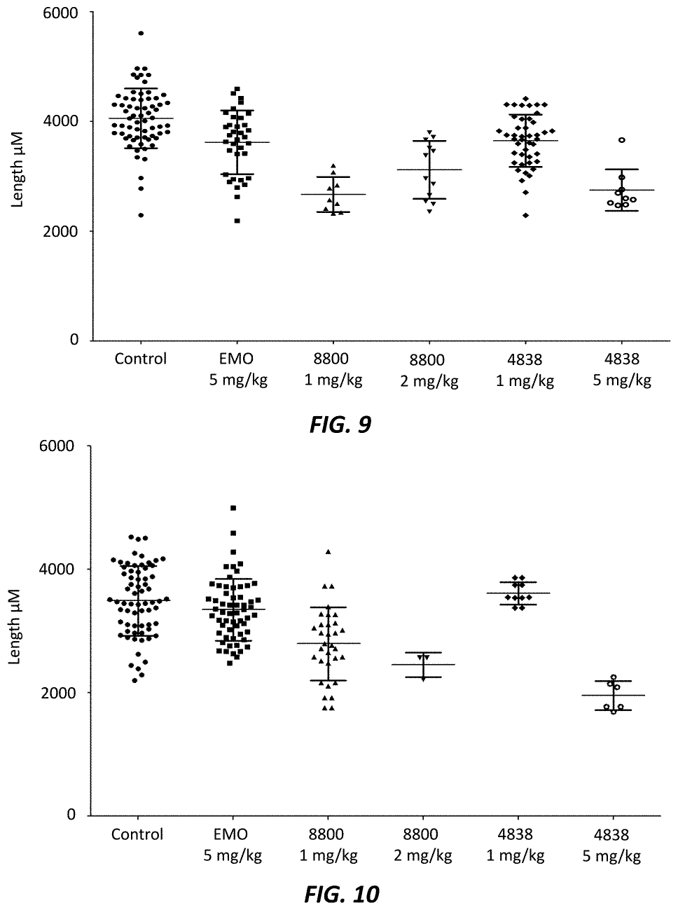

In another example, NSG mice were infected with 50 Di L3 (UC strain, sourced from Merial's insectary) per mouse, then treated on Days 1, 15, and 30 according to the following: EMO (5 mg/kg); 8800 (1 mg/kg); 8800 (2 mg/kg); 4838 (1 mg/kg); or 4838 (5 mg/kg). FIG. 9 is a graph showing the lengths of Di recovered at Days 41 to 42, and Table 12 presents the survival data, recovery data (Days 41 and 42), and summary statistics.

TABLE 12
Summary data for Di-infected NSG mice treated with Control, EMO, 8800, or 4838.

| | | | EMO High Dose- | | | 8800 Normal Dose- | | | 8800 High Dose- | | | 4838 Normal Dose- | | | 4838 High Dose- | | |
|---|---|---|---|---|---|---|---|---|---|---|---|---|---|---|---|---|---|
| Control | | | 5 mg/kg | | | 1 mg/kg | | | 2 mg/kg | | | 1 mg/kg | | | 5 mg/kg | | |
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 19 | 38 | 8 | 7 | 14 | 22 | 5 | 10 | 15 | 4 | 8 | 29 | 8 | 16 | 36 | 1 | 2 |
| 2 | 19 | 38 | 9 | 11 | 22 | 23 | 7 | 14 | 16 | 1 | 2 | 30 | 7 | 14 | 37 | 2 | 4 |
| 3 | 12 | 24 | 10 | 10 | 20 | 24 | 3 | 6 | 17 | 1 | 2 | 31 | 7 | 14 | 38 | 2 | 4 |
| 4 | 22 | 44 | 11 | 11 | 22 | 25 | 2 | 4 | 18 | 1 | 2 | 32 | 8 | 16 | 39 | 0 | 0 |
| 5 | 16 | 32 | 12 | 10 | 20 | 26 | 1 | 2 | 19 | 0 | 0 | 33 | 9 | 18 | 40 | 1 | 2 |
| 6 | 19 | 38 | 13 | 10 | 20 | 27 | 4 | 8 | 20 | 0 | 0 | 34 | 7 | 14 | 41 | 2 | 4 |
| 7 | 19 | 38 | 14 | 9 | 18 | 28 | 3 | 6 | 21 | 0 | 0 | 35 | 8 | 16 | 42 | 2 | 4 |
| Mean | 18 | 36 | Mean | 10 | 19 | Mean | 4 | 7 | Mean | 1 | 2 | Mean | 8 | 15 | Mean | 1 | 3 |
| SD | 3 | 6 | SD | 1 | 3 | SD | 2 | 4 | SD | 1 | 3 | SD | 1 | 2 | SD | 1 | 2 |

| | | | | | | | | | |
|---|---|---|---|---|---|---|---|---|---|
| % | 47% | % | 81% | % | 94% | % | 58% | % | 92% |
| Red | | Red | | Red | | Red | | Red | |
| P | 0.000 | P | 0.000 | P | 0.000 | P | 0.000 | P | 0.000 |
| value | | value | | value | | value | | value | |

In another example, NSG mice were infected with 50 Di L3 (UC Strain) per mouse, then treated on Days 1, 15, and 30 according to the following: EMO (1 mg/kg); 8800 (1 mg/kg); 8800 (2 mg/kg); 4838 (1 mg/kg); or 4838 (5 mg/kg). FIG. 10 is a graph showing the lengths of Di recovered at Days 40 to 42, and Table 13 presents the survival data, recovery data (Days 41 to 43), and summary statistics.

TABLE 13
Summary data for Di-infected NSG mice treated with Control, EMO, 8800 or 4838.

| Control | | | EMO 1 mg/kg | | | 8800 1 mg/kg | | | 8800 2 mg/kg | | | 4838 1 mg/kg | | | 4838 5 mg/kg | | |
|---|---|---|---|---|---|---|---|---|---|---|---|---|---|---|---|---|---|
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 20 | 40 | 8 | 6 | 12 | 11 | 2 | 4 | 14 | 0 | 0 | 18 | 3 | 6 | 17 | 1 | 2 |
| 2 | 14 | 28 | 9 | 15 | 30 | 12 | 3 | 6 | 15 | 1 | 2 | 19 | 1 | 2 | 35 | 2 | 4 |
| 3 | 21 | 42 | 10 | 18 | 36 | 13 | 6 | 12 | 16 | 2 | 4 | 20 | 3 | 6 | 36 | 1 | 2 |
| 4 | 15 | 30 | 33 | 9 | 18 | 29 | 5 | 10 | 25 | 1 | 2 | 21 | 5 | 10 | 37 | 1 | 2 |
| 5 | 19 | 38 | 34 | 13 | 26 | 30 | 7 | 14 | 26 | 0 | 0 | 22 | 7 | 14 | 40 | 1 | 2 |
| 6 | 24 | 48 | 38 | 9 | 18 | 31 | 5 | 10 | 27 | 3 | 6 | 23 | 4 | 8 | 41 | 0 | 0 |
| 7 | 20 | 40 | 39 | 15 | 30 | 32 | 8 | 16 | 28 | 1 | 2 | 24 | 10 | 20 | 42 | 2 | 4 |
| Mean | 19 | 38 | Mean | 12 | 24 | Mean | 5 | 10 | Mean | 1 | 2 | Mean | 5 | 9 | Mean | 1 | 2 |
| SD | 3 | 7 | SD | 4 | 9 | SD | 2 | 4 | SD | 1 | 2 | SD | 3 | 6 | SD | 1 | 1 |

| | | | | | | | | | |
|---|---|---|---|---|---|---|---|---|---|
| % | 37% | % | 73% | % | 94% | % | 75% | % | 94% |
| Red | | Red | | Red | | Red | | Red | |
| P | 0.000 | P | 0.000 | P | 0.000 | P | 0.000 | P | 0.000 |
| value | | value | | value | | value | | value | |

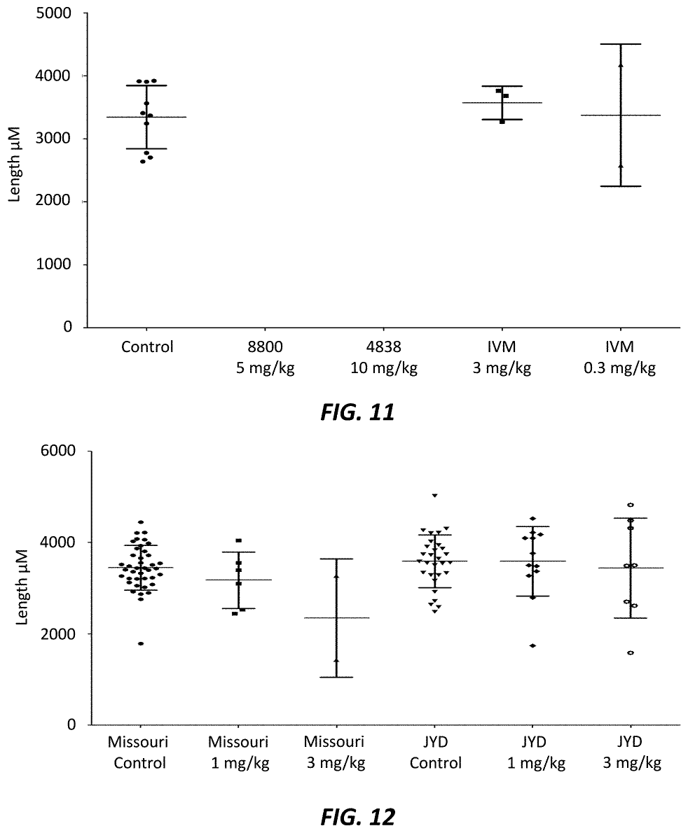

In another example, NSG mice were infected with 50 Di L3 per mouse, then treated on Days 1, 15, and 30 according to the following: 8800 (5 mg/kg); 4838 (10 mg/kg); IVM (3 mg/kg); or IVM (0.3 mg/kg). FIG. 11 is a graph showing the lengths of Di recovered at Days 41 to 42, and Table 14 presents the survival data, recovery data, and summary statistics.

TABLE 14
Summary data for Di-infected NSG mice treated with Control, 8800, 4838, or IVM.

| Control | | | 8800 5 mg/kg | | | 4838 10 mg/kg | | | IVM 3 mg/kg | | | IVM 0.3 mg/kg | | |
|---|---|---|---|---|---|---|---|---|---|---|---|---|---|---|
| | | % | | | % | | | % | | | % | | | % |
| # | Survival | Rec | # | Survival | Rec | # | Survival | Rec | # | Survival | Rec | # | Survival | Rec |
| 1 | 7 | 14 | 6 | 0 | 0 | 11 | 0 | 0 | 16 | 0 | 0 | 21 | 1 | 2 |
| 2 | 9 | 18 | 7 | 0 | 0 | 12 | 0 | 0 | 17 | 1 | 2 | 22 | 0 | 0 |
| 3 | 2 | 4 | 8 | 0 | 0 | 13 | 0 | 0 | 18 | 1 | 2 | 23 | 0 | 0 |
| 4 | 8 | 16 | 9 | 0 | 0 | 14 | 0 | 0 | 19 | 0 | 0 | 24 | 0 | 0 |
| 5 | 8 | 16 | 10 | 0 | 0 | 15 | 0 | 0 | 20 | 1 | 2 | 25 | 1 | 2 |
| Mean | 7 | 14 | Mean | 0 | 0 | Mean | 0 | 0 | Mean | 1 | 1 | Mean | 0 | 1 |
| SD | 3 | 6 | SD | 0 | 0 | SD | 0 | 0 | SD | 1 | 1 | SD | 1 | 1 |

| | | | | | | | |
|---|---|---|---|---|---|---|---|
| % | 100% | % | 100% | % | 93% | % | 93% |
| Red | | Red | | Red | | Red | |
| P | 0.000 | P | 0.000 | P | 0.000 | P | 0.000 |
| value | | value | | value | | value | |

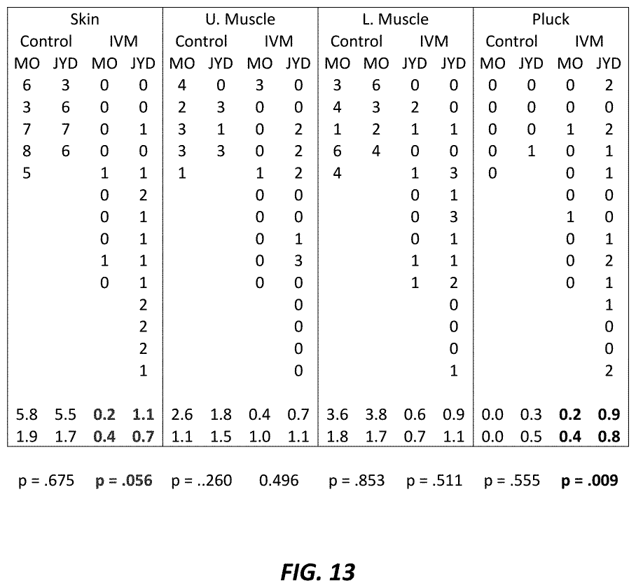

In another example, NSG mice were infected on Day 0 with 50 Di L3 (FR3 worms) per mouse, then treated on Days 1, 15, and 30 according to the following: MO Control; MO IVM (1 mg/kg); MO IVM (3 mg/kg); JYD Control; JYD IVM (1 mg/kg); JYD IVM (3 mg/kg); 4838 (10 mg/kg); IVM (3 mg/kg); or IVM (0.3 mg/kg). We used either the “ivermectin-susceptible” Missouri strain or the “ivermectin-resistant” JYD strain. The Missouri strain (i.e., Missouri isolate) and the JYD strain (i.e., isolate JYD-27) are described, for example, in Maclean et al. (2017) Parasit. Vectors 10(Suppl 2):480, herein incorporated by reference in its entirety for all purposes. The Missouri strain is also described, for example, in Bourguinat et al. (2011) Vet. Parasitol. 176(4):368-373, herein incorporated by reference in its entirety for all purposes. FIG. 12 is a graph showing the lengths of Di recovered at Days 41 to 42, and Table 15 presents the survival data, recovery data (Days 41 and 42), and summary statistics. FIG. 13 shows the worm recovery locations.

TABLE 15
Summary data for Di-infected NSG mice treated with Control, 8800, 4838, or IVM.

| Group 1 | | | Group 2 | | | Group 3 | | | Group 4 | | | Group 5 | | | Group 6 | | |
|---|---|---|---|---|---|---|---|---|---|---|---|---|---|---|---|---|---|
| Missouri | | | Missouri IVM | | | Missouri IVM | | | JYD | | | JYD IVM | | | JYD IVM | | |
| Control | | | 1 mg/kg | | | 3 mg/kg | | | Control | | | 1 mg/kg | | | 3 mg/kg | | |
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 13 | 26 | 6 | 3 | 6 | 11 | 0 | 0 | 16 | 9 | 18 | 23 | 6 | 12 | 21 | 2 | 4 |
| 2 | 18 | 36 | 7 | 2 | 4 | 12 | 1 | 2 | 17 | 12 | 24 | 24 | 3 | 6 | 22 | 0 | 0 |
| 3 | 22 | 44 | 8 | 2 | 4 | 13 | 0 | 0 | 18 | 10 | 20 | 25 | 7 | 14 | 29 | 7 | 14 |
| 4 | 34 | 68 | 9 | 0 | 0 | 14 | 2 | 4 | 20 | 14 | 28 | 26 | 3 | 6 | 30 | 4 | 8 |
| 5 | 20 | 40 | 10 | 3 | 6 | 15 | 1 | 2 | | | | 27 | 4 | 8 | 31 | 3 | 6 |
| | | | | | | | | | | | | 28 | 4 | 8 | 32 | 2 | 4 |
| | | | | | | | | | | | | 34 | 4 | 8 | 33 | 2 | 4 |
| Mean | 21 | 43 | Mean | 2 | 4 | Mean | 1 | 2 | Mean | 11 | 23 | Mean | 4 | 9 | Mean | 3 | 6 |
| SD | 8 | 16 | SD | 1 | 2 | SD | 1 | 2 | SD | 2 | 4 | SD | 2 | 3 | SD | 2 | 4 |

| % Red: 91% | | % Red: 95% | | % Red | % Red: 61% | | % Red: 74% | |
|---|---|---|---|---|---|---|---|---|
| P value | 0.000 | P | 0.000 | P value | P | 0.003 | P value | 0.000 |
| | | value | | | value | | | |

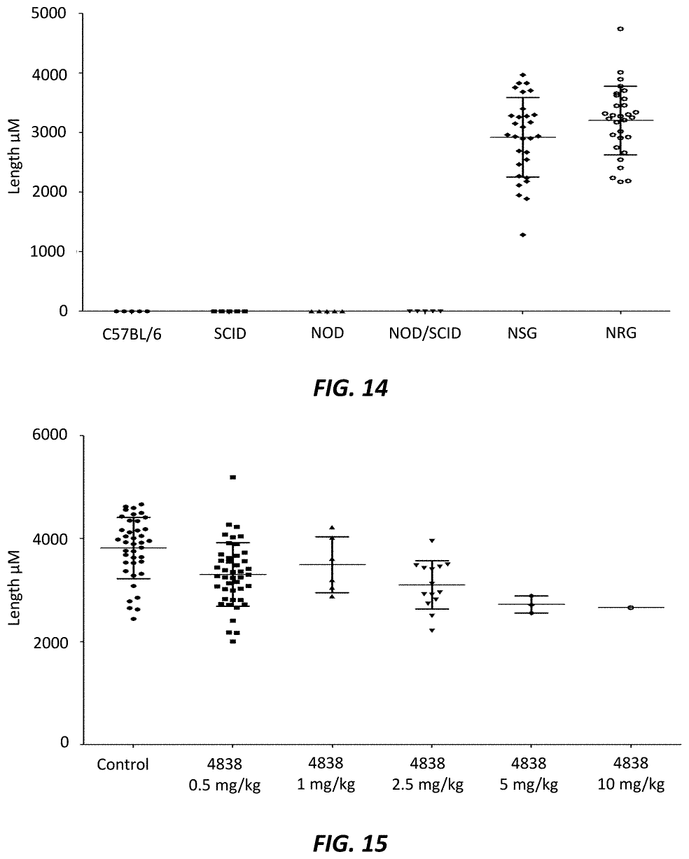

In another example, various strains of mice were infected on Day 0 with 50 Di L3 (FR3 worms) per mouse, but not treated subsequently. FIG. 14 is a graph showing the lengths of Di recovered at Days 41 to 42, and Table 16 presents the survival data, recovery data (Days 41 and 42), and summary statistics.

TABLE 16
Summary data for various strains of mouse infected with FR3 Di.

| C57BL/6 | | | SCID | | | NOD | | | NOD/SCID | | | NSG | | | NRG | | |
|---|---|---|---|---|---|---|---|---|---|---|---|---|---|---|---|---|---|
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 0 | 0 | 6 | 0 | 0 | 11 | 0 | 0 | 16 | 0 | 0 | 21 | 10 | 20 | 26 | 13 | 26 |
| 2 | 0 | 0 | 7 | 0 | 0 | 12 | 0 | 0 | 17 | 0 | 0 | 22 | 17 | 34 | 27 | 8 | 16 |
| 3 | 0 | 0 | 8 | 0 | 0 | 13 | 0 | 0 | 18 | 0 | 0 | 23 | 21 | 42 | 28 | 7 | 14 |
| 4 | 0 | 0 | 9 | 0 | 0 | 14 | 0 | 0 | 19 | 0 | 0 | 24 | 12 | 24 | 29 | 10 | 20 |
| 5 | 0 | 0 | 10 | 0 | 0 | 15 | 0 | 0 | 20 | 0 | 0 | 25 | 13 | 26 | 30 | 15 | 30 |
| Mean | 0 | 0 | Mean | 0 | 0 | Mean | 0 | 0 | Mean | 0 | 0 | Mean | 15 | 29 | Mean | 11 | 21 |
| SD | 0 | 0 | SD | 0 | 0 | SD | 0 | 0 | SD | 0 | 0 | SD | 4 | 9 | SD | 3 | 7 |

In another example, NSG mice were infected on Day 0 with 50 Di L3 (FR3 worms) per mouse, and treated on days 1, 15, and 30 as follows: 4838 (0.5 mg/kg); 4838 (1 mg/kg); 4838 (2.5 mg/kg); 4838 (5 mg/kg); 4838 (10 mg/kg). FIG. 15 is a graph showing the lengths of Di recovered at Days 41 to 42, and Table 17 presents the survival data, recovery data (Days 41 and 42), and summary statistics.

TABLE 17
Summary data for NSG mice infected with FR3 Di and subsequently treated with increasing doses of the 4838 depsipeptide
parasiticidal agent.

| Control | | | 4838 .5 mg/kg | | | 4838 1 mg/kg | | | 4838 2.5 mg/kg | | | 4838 5 mg/kg | | | 4838 10 mg/kg | | |
|---|---|---|---|---|---|---|---|---|---|---|---|---|---|---|---|---|---|
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 14 | 28 | 8 | 6 | 12 | 15 | 5 | 10 | 22 | 2 | 4 | 29 | 0 | 0 | 36 | 0 | 0 |
| 2 | 17 | 34 | 9 | 14 | 28 | 16 | 6 | 12 | 23 | 4 | 8 | 30 | 2 | 4 | 37 | 0 | 0 |
| 3 | 15 | 30 | 10 | 11 | 22 | 17 | 5 | 10 | 24 | 1 | 2 | 31 | | 0 | 38 | 0 | 0 |
| 4 | 13 | 26 | 11 | 4 | 8 | 18 | 10 | 20 | 25 | 6 | 12 | 32 | 0 | 0 | 39 | 0 | 0 |
| 5 | 16 | 32 | 12 | 4 | 8 | 19 | 8 | 16 | 26 | 2 | 4 | 33 | 2 | 4 | 40 | 0 | 0 |
| 6 | 8 | 16 | 13 | 17 | 34 | 20 | 8 | 16 | 27 | 5 | 10 | 34 | 2 | 4 | 41 | 1 | 2 |
| 7 | 14 | 28 | 14 | 8 | 16 | 21 | 6 | 12 | 28 | 2 | 4 | 35 | 0 | 0 | 42 | 0 | 0 |
| Mean | 14 | 28 | Mean | 9 | 18 | Mean | 7 | 14 | Mean | 3 | 6 | Mean | 1 | 2 | Mean | 0 | 0.29 |
| SD | 3 | 6 | SD | 5 | 10 | SD | 2 | 4 | SD | 2 | 4 | SD | 1 | 2 | SD | 0 | 1 |

| % Red: 36% | | % Red: 50% | | % Red: 76% | | % Red: 93% | | % Red: 99% | |
|---|---|---|---|---|---|---|---|---|---|
| P value | 0.002 | P | 0.000 | P value | 0.000 | P | 0.000 | P value | 0.000 |
| | | value | | | | value | | | |

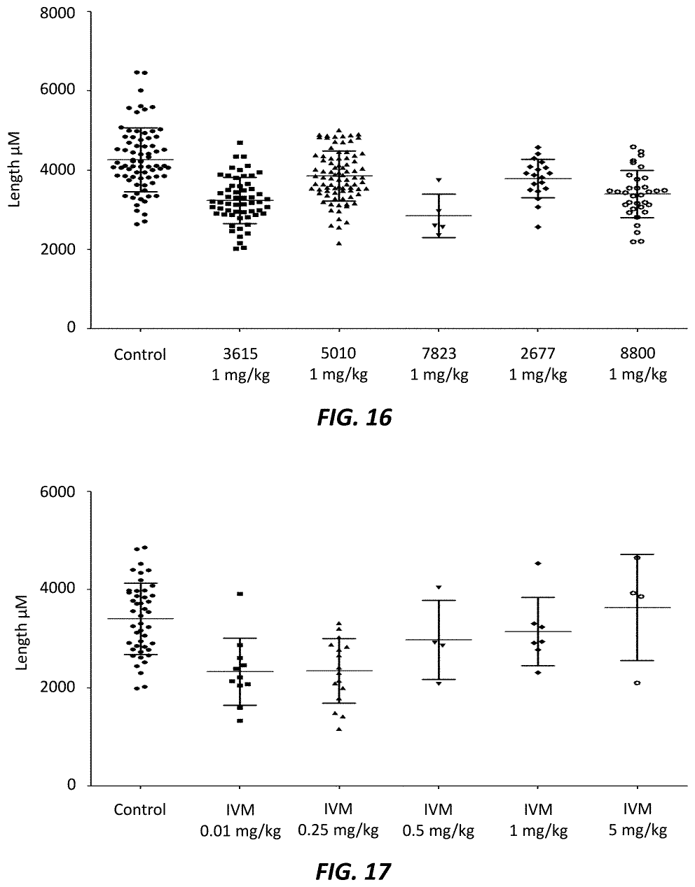

In another example, NSG mice were infected on Day 0 with 50 JYD Di L3 (FR3 worms) per mouse, then treated on Days 1, 15, and 30 according to the following: Control; 3615 (1 mg/kg); 5010 (1 mg/kg); 7823 (1 mg/kg); 2677 (1 mg/kg); or 8800 (1 mg/kg). FIG. 16 is a graph showing the lengths of Di recovered at Days 41 to 42, and Table 18 presents the survival data, recovery data (Days 41 and 42), and summary statistics.

TABLE 18
Summary data for NSG mice infected with FR3 Di and subsequently treated with 3615, 5010, 7823, 2677, and 8800.

| Control | | | 3615 1 mg/kg | | | 5010 1 mg/kg | | | 7823 1 mg/kg | | | 2677 1 mg/kg | | | 8800 1 mg/kg | | |
|---|---|---|---|---|---|---|---|---|---|---|---|---|---|---|---|---|---|
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 21 | 42 | 8 | 14 | 28 | 15 | 17 | 34 | 22 | 2 | 4 | 29 | 9 | 18 | 36 | 2 | 4 |
| 2 | 23 | 46 | 9 | 18 | 36 | 16 | 15 | 30 | 23 | 1 | 2 | 30 | 8 | 16 | 37 | 13 | 26 |
| 3 | 20 | 40 | 10 | 8 | 16 | 17 | 19 | 38 | 24 | 0 | 0 | 31 | 2 | 4 | 38 | 9 | 18 |
| 4 | 24 | 48 | 11 | 12 | 24 | 18 | 12 | 24 | 25 | 1 | 2 | 32 | 3 | 6 | 39 | 6 | 12 |
| 5 | 12 | 24 | 12 | 13 | 26 | 19 | 18 | 36 | 26 | 0 | 0 | 33 | 2 | 4 | 40 | 8 | 16 |
| 6 | 25 | 50 | 13 | 15 | 30 | 20 | 12 | 24 | 27 | 0 | 0 | 34 | 2 | 4 | 41 | 6 | 12 |
| 7 | 24 | 48 | 14 | 12 | 24 | 21 | 7 | 14 | 28 | 0 | 0 | 35 | 1 | 2 | 42 | 4 | 8 |
| Mean | 21 | 43 | Mean | 13 | 26 | Mean | 14 | 29 | Mean | 1 | 1 | Mean | 4 | 8 | Mean | 7 | 14 |
| SD | 4 | 9 | SD | 3 | 6 | SD | 4 | 8 | SD | 1 | 2 | SD | 3 | 6 | SD | 4 | 7 |

| % Red: 40% | | % Red: 33% | | % Red: 98% | | % Red: 81% | | % Red: 67% | |
|---|---|---|---|---|---|---|---|---|---|
| P value | 0.000 | P | 0.001 | P value | 0.000 | P | 0.000 | P value | 0.000 |
| | | value | | | | value | | | |

In another example, NSG mice were infected on Day 0 with 50 JYD Di L3 (FR3 worms) per mouse, then treated on Days 1, 15, and 30 according to the following: Control; IVM (0.01 mg/kg); IVM (0.25 mg/kg); IVM (0.5 mg/kg); IVM (1 mg/kg); or IVM (5 mg/kg). FIG. 17 is a graph showing the lengths of Di recovered at Days 40 to 42, and Table 19 presents the survival data, recovery data (Days 40-42), and summary statistics.

TABLE 19
Summary data for NSG mice infected with FR3 Di and subsequently treated with different doses of IVM.

| Control | | | IVM 0.01 mg/kg | | | IVM 0.25 mg/kg | | | IVM 0.5 mg/kg | | | IVM 1 mg/kg | | | IVM 5 mg/kg | | |
|---|---|---|---|---|---|---|---|---|---|---|---|---|---|---|---|---|---|
| | | % | | | % | | | % | | | % | | | % | | | % |
| # | Survival | Rec | # | Survival | Rec | # | Survival | Rec | # | Survival | Rec | # | Survival | Rec | # | Survival | Rec |
| 1 | 8 | 16 | 8 | 3 | 6 | 15 | 2 | 4 | 22 | 0 | 0 | 29 | 1 | 2 | 36 | 0 | 0 |
| 2 | 9 | 18 | 9 | 4 | 8 | 16 | 0 | 0 | 23 | 4 | 8 | 30 | 1 | 2 | 37 | 1 | 2 |
| 3 | 10 | 20 | 10 | 3 | 6 | 17 | 3 | 6 | 24 | 1 | 2 | 31 | 4 | 8 | 38 | 2 | 4 |
| 4 | 10 | 20 | 11 | 2 | 4 | 18 | 2 | 4 | 25 | 1 | 2 | 32 | 1 | 2 | 39 | 3 | 6 |
| 5 | 2 | 4 | 12 | 1 | 2 | 19 | 7 | 14 | 26 | 0 | 0 | 33 | 2 | 4 | 40 | 0 | 0 |
| 6 | 12 | 24 | 13 | 3 | 6 | 20 | 5 | 10 | 27 | 0 | 0 | 34 | 0 | 0 | 41 | 0 | 0 |
| 7 | 13 | 26 | 14 | 0 | 0 | 21 | 5 | 10 | 28 | 2 | 4 | 35 | 0 | 0 | 42 | 0 | 0 |
| Mean | 9 | 18 | Mean | 2 | 5 | Mean | 3 | 7 | Mean | 1 | 2 | Mean | 1 | 3 | Mean | 1 | 2 |
| SD | 4 | 7 | SD | 1 | 3 | SD | 2 | 5 | SD | 1 | 3 | SD | 1 | 3 | SD | 1 | 2 |

| % Red: 72% | | % Red: 61% | | % Red: 89% | | % Red: 83% | | % Red: 89% | |
|---|---|---|---|---|---|---|---|---|---|
| P value | 0.000 | P | 0.000 | P value | 0.000 | P | 0.000 | P value | 0.000 |
| | | value | | | | value | | | |

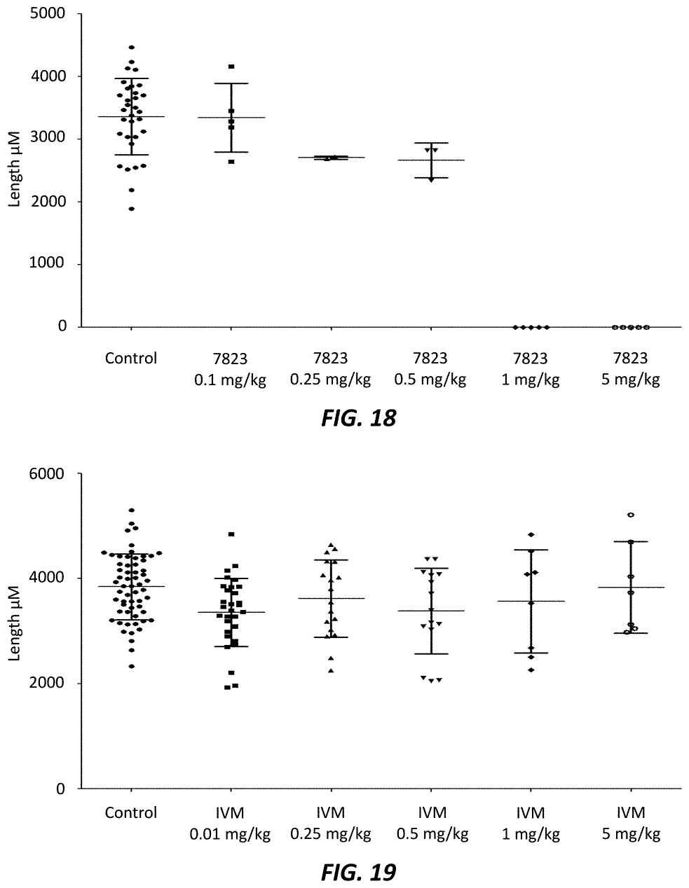

In another example, NSG mice were infected on Day 0 with 50 JYD Di L3 (FR3 worms) per mouse, then treated on Days 1, 15, and 30 according to the following: Control; 7823 (0.1 mg/kg); 7823 (0.25 mg/kg); 7823 (0.5 mg/kg); 7823 (1 mg/kg); or 7823 (5 mg/kg). FIG. 18 is a graph showing the lengths of Di recovered at Days 40 to 42, and Table 20 presents the survival data, recovery data (Days 40-42), and summary statistics.

TABLE 20
Summary data for NSG mice infected with FR3 Di and subsequently treated with different doses of 7823.

| Control | | | 7823 0.1 mg/kg | | | 7823 0.25 mg/kg | | | | | | | | | | | |
|---|---|---|---|---|---|---|---|---|---|---|---|---|---|---|---|---|---|
| | | % | | | % | | | % | 7823 0.5 mg/kg | | | 7823 1.0 mg/kg | | | 7823 5.0 mg/kg | | |
| # | Survival | Rec | # | Survival | Rec | # | Survival | Rec | # | Survival | % Rec | # | Survival | % Rec | # | Survival | % Rec |
| 1 | 11 | 22 | 6 | 4 | 8 | 11 | 2 | 4 | 16 | 0 | 0 | 21 | 0 | 0 | 26 | 0 | 0 |
| 2 | 9 | 18 | 7 | 1 | 2 | 12 | 0 | 0 | 17 | 2 | 4 | 22 | 0 | 0 | 27 | 0 | 0 |
| 3 | 14 | 28 | 8 | 3 | 6 | 13 | 0 | 0 | 18 | 1 | 2 | 23 | 0 | 0 | 28 | 0 | 0 |
| 4 | 11 | 22 | 9 | 3 | 6 | 14 | 2 | 4 | 19 | 0 | 0 | 24 | 0 | 0 | 29 | 0 | 0 |
| 5 | 13 | 26 | 10 | 0 | 0 | 15 | 0 | 0 | 20 | 4 | 8 | 25 | 0 | 0 | 30 | 0 | 0 |
| Mean | 12 | 23 | Mean | 2 | 4 | Mean | 1 | 2 | Mean | 1 | 3 | Mean | 0 | 0 | Mean | 0 | 0 |
| SD | 2 | 4 | SD | 2 | 3 | SD | 1 | 2 | SD | 2 | 3 | SD | 0 | 0 | SD | 0 | 0 |

| % Red: 83% | | % Red: 91% | | % Red: 87% | | % Red: 100% | | % Red: 100% | |
|---|---|---|---|---|---|---|---|---|---|
| P | 0.000 | P | 0.000 | P | 0.000 | P | 0.000 | P | 0.000 |
| value | | value | | value | | value | | value | |

In another example, NSG mice were infected on Day 0 with 50 JYD Di L3 (FR3 worms) per mouse, then treated on Days 1, 15, and 30 according to the following: Control; IVM (0.01 mg/kg); IVM (0.25 mg/kg); IVM (0.5 mg/kg); IVM (1 mg/kg); or IVM (5 mg/kg). FIG. 19 is a graph showing the lengths of Di recovered at Days 40 to 42, and Table 21 presents the survival data, recovery data (Days 40-42), and summary statistics.

TABLE 21
Summary data for NSG mice infected with FR3 Di and subsequently treated with different doses of IVM.

| Control | | | IVM 0.01 mg/kg | | | IVM 0.25 mg/kg | | | | | | | | | | | |
|---|---|---|---|---|---|---|---|---|---|---|---|---|---|---|---|---|---|
| | | % | | | % | | | % | IVM 0.5 mg/kg | | | IVM 1 mg/kg | | | IVM 5 mg/kg | | |
| # | Survival | Rec | # | Survival | Rec | # | Survival | Rec | # | Survival | % Rec | # | Survival | % Rec | # | Survival | % Rec |
| 1 | 14 | 28 | 8 | 6 | 12 | 15 | 0 | 0 | 22 | 2 | 4 | 29 | 3 | 6 | 36 | 3 | 6 |
| 2 | 8 | 16 | 9 | 4 | 8 | 16 | 3 | 6 | 23 | 6 | 12 | 30 | 3 | 6 | 37 | 2 | 4 |
| 3 | 11 | 22 | 10 | 8 | 16 | 17 | 2 | 4 | 24 | 1 | 2 | 31 | 0 | 0 | 38 | 2 | 4 |
| 4 | 15 | 30 | 11 | 7 | 14 | 18 | 5 | 10 | 25 | 3 | 6 | 32 | 1 | 2 | 39 | 0 | 0 |
| 43 | 18 | 36 | 12 | 9 | 18 | 19 | 4 | 8 | 26 | 0 | 0 | 33 | 2 | 4 | 40 | 3 | 6 |
| 5 | 10 | 20 | 13 | 5 | 10 | 20 | 4 | 8 | 27 | 6 | 12 | 34 | 1 | 2 | 41 | 2 | 4 |
| 6 | 13 | 26 | 14 | 7 | 14 | 21 | 5 | 10 | 28 | 4 | 8 | 35 | 1 | 2 | 42 | 4 | 8 |
| 7 | 6 | 12 | | | | | | | | | | | | | | | |
| Mean | 12 | 24 | Mean | 7 | 13 | Mean | 3 | 7 | Mean | 3 | 6 | Mean | 2 | 3 | Mean | 2 | 5 |
| SD | 4 | 8 | SD | 2 | 3 | SD | 2 | 4 | SD | 2 | 5 | SD | 1 | 2 | SD | 1 | 3 |

| % Red: 46% | | % Red: 71% | | % Red: 75% | | % Red: 88% | | % Red: 79% | |
|---|---|---|---|---|---|---|---|---|---|
| P | 0.000 | P | 0.000 | P | 0.000 | P | 0.000 | P | 0.000 |
| value | | value | | value | | value | | value | |

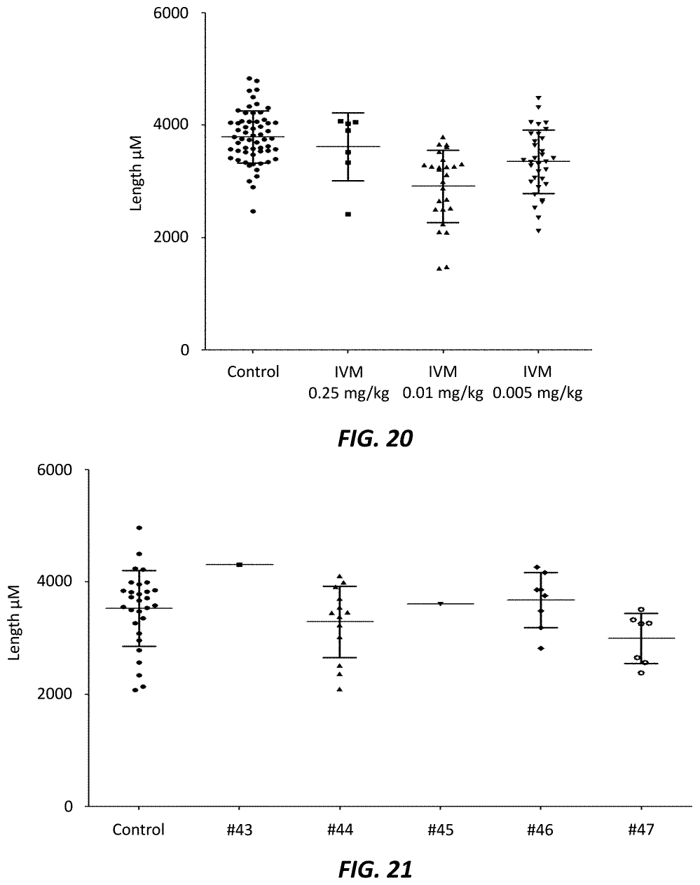

In another example, NSG mice were infected on Day 0 with 50 JYD Di L3 (FR3 worms) per mouse, then treated on Days 1, 15, and 30 according to the following: Control; IVM (0.25 mg/kg); IVM (0.01 mg/kg); or IVM (0.005 mg/kg). FIG. 20 is a graph showing the lengths of Di recovered at Days 40 to 42, and Table 22 presents the survival data, recovery data (Days 40-42), and summary statistics.

TABLE 22
Summary data for NSG mice infected with FR3 Di and subsequently treated with IVM.

| Control | | | IVM 0.25 mg/kg | | | IVM 0.01 mg/kg | | | IVM 0.005 mg/kg | | |
|---|---|---|---|---|---|---|---|---|---|---|---|
| | | % | | | % | | | % | | | % |
| # | Survival | Rec | # | Survival | Rec | # | Survival | Rec | # | Survival | Rec |
| 1 | 14 | 28 | 8 | 6 | 12 | 15 | 6 | 12 | 22 | 6 | 12 |
| 2 | 16 | 32 | 9 | 4 | 8 | 16 | 3 | 6 | 23 | 7 | 14 |
| 3 | 15 | 30 | 10 | 0 | 0 | 17 | 2 | 4 | 24 | 7 | 14 |
| 4 | 15 | 30 | 11 | 1 | 2 | 18 | 4 | 8 | 25 | 6 | 12 |
| 5 | 15 | 30 | 12 | 0 | 0 | 19 | 2 | 4 | 26 | 5 | 10 |
| 6 | 6 | 12 | 13 | 3 | 6 | 20 | 2 | 4 | 27 | 5 | 10 |
| 7 | 10 | 20 | 14 | 2 | 4 | 21 | 6 | 12 | 28 | 6 | 12 |
| Mean | 13 | 26 | Mean | 2 | 5 | Mean | 4 | 7 | Mean | 6 | 12 |
| SD | 4 | 7 | SD | 2 | 4 | SD | 2 | 4 | SD | 1 | 2 |

| % Red: 81% | | % Red: 73% | | % Red: 54% | |
|---|---|---|---|---|---|
| P | 0.000 | P | 0.000 | P | 0.000 |
| value | | value | | value | |

A single experiment was performed in which NSG mice were infected with approximately 200,000 JYD strain D. immitis microfilariae by retro-orbital infection. Microfilariae were consistently recovered from the retro-orbital blood at 4 weeks post-infection. See Table 23. Through this experiment we demonstrated that the NSG mouse was also a suitable host for the microfilarial stage of D. immitis.

TABLE 23
Infection of NSG mice with Di microfilariae.

| | Mf Count/100 μL Blood | | |
|---|---|---|---|
| Mouse ID | 2 Week | 4 Week | 6 Week |
| 1R | 7 | 3 | 0 |
| 1L | 10 | 4 | X |
| RL | 12 | 4 | 1 |
| NT | 6 | 2 | 0 |
| 2R2L | 12 | 10 | 0 |
| 2R | 8 | 6 | 2 |

### Example 2—Infection of Immunocompromised Mice with Ivermectin-Resistant (IVMR) Strains of D. immitis to Screen for Novel Compounds to Address Resistant Strains

NSG mice infected with resistant HW are exposed to novel compounds to determine the efficacy of these compounds in killing resistant parasites. The source of the novel compounds may be from pharmaceutical libraries and they would be administered to the mice in a range of doses to measure efficacy and killing IVM-resistant and IVM-susceptible strains of HW.

One such strain that is resistant (i.e., less susceptible) to ivermectin is the JYD strain described above. The JYD strain (i.e., isolate JYD-27) is described, for example, in Maclean et al. (2017) Parasit. Vectors 10(Suppl 2):480, herein incorporated by reference in its entirety for all purposes. Experiments in which compounds were tested against the JYD strain in NSG immunocompromised mice are described in Example 1. See, e.g., FIGS. 12, 13, and 16-20 and Tables 15 and 18-22. See also FIG. 21 and Table 25 in Example 3.

### Example 3—Development and Validation of an Immunocompetent, Intermediate In Vivo Model of Canine Heartworm Infection with Engrafted Canine Immune Cells

We performed an initial experiment using peripheral blood mononuclear cells (PBMCs) purified from whole blood. Canine PBMCs were isolated from 10 mL of whole blood using a Ficoll gradient per manufacturer's instructions for the isolation of human PBMC. Six NSG mice were injected via intraperitoneal (IP) injection with 5.0×10⁶ PBMCs from a single donor animal and screened by flow cytometry 4 and 8 weeks later. At 4 weeks post-engraftment, all mice were positive for canine B cells and CD4 and CD8 T cells, along with a number of other canine CD45⁺ cells present that the panel did not identify (Table 24 and data not shown). Percent engraftment was calculated by the total canine CD45+ cells within the lymphocyte/granulocyte gate, which is determined based on cell size and cell granularity. At 8 weeks post-engraftment 3 mice had succumbed to graft vs host disease (GVHD), and of the remaining 3 mice, only one had significant levels of canine PBMCs remaining. This study shows that NSG mice are capable of supporting canine immune cells and should provide a suitable platform for the engraftment of CD34⁺ stem cells.

TABLE 24
Canine cell levels in NSG mice at 3-weeks post-engraftment.

| CD45+ | CD3+ | CD4+ | CD8+ |
|---|---|---|---|
| 29 ± 20% | 8 ± 7% | 16 ± 14% | 3 ± 3% |

To further confirm the initial PBMC findings, PBMCs were purified from 100 mL of canine whole blood. Fifteen NSG mice were injected with either 5×10⁶ (10 mice) or 10×10⁶ (5 mice) PBMCs from a donor animal. Mice receiving 5×10⁶ cells had an average canine CD45⁺ engraftment of 16±16%, and the mice receiving 10×10⁶ cells had an average engraftment of 28±13%. All mice succumbed to GVHD between 3 and 6 weeks post-engraftment.

Next, two groups of CD34+ stem cells were purified from two different canine umbilical cord blood collections. Canine CD34⁺ stem cells were purified using a biotin labeled anti-canine CD34⁺ antibody, in conjunction with a magnetic assisted cell sorting kit (MACS) that specifically recognized the biotin labeled antibody. Due to the small amount of cord blood obtained from a puppy, it is impossible to purify stem cells from a single donor. Two mixed-donor purified CD34⁺ cells batches were purified with cell counts of 2.65×10⁵ and 2.2×10⁵ and cell purities of 74 and 77%, respectively. 50,000 canine CD34+ cells isolated from umbilical cord blood were then engrafted into five 48-hour-old NSG pups irradiated with 150 RAD. Their engraftment status was verified at 8 weeks. Based on our experience with humanized mice, it was predicted that the mice would be optimally engrafted at around 12 weeks. At 12 weeks post-engraftment, the 5 mice had average canine CD45⁺ cell levels of 58±6%. The caninized mice (CnMice) were then infected with 50 JYD Di L3 about one week later (Day 0) in conjunction with an NSG mouse infection. The mice were necropsied on Days 40 through 42 post-infection. It was concluded that there was no difference in the parasite survival and growth in the CnMice vs. the NSG mice. FIG. 21 is a graph showing the lengths of Di recovered at Days 40 to 42 in the canine mice, and Table 25 presents the survival and recovery data (Days 40-42). There was no difference in the parasite survival and growth in the CnMice vs. the NSG mice.

TABLE 25
Summary data and recovery of Di for CnMice and NSG
control mice infected with FR3 Di

| | | | U. | L. | | | % | Average % |
|---|---|---|---|---|---|---|---|---|
| | Mouse # | Skin | Muscle | Muscle | Pluck | Total | Recovery | Recovery |
| NSG | 1 | 4 | 0 | 5 | 0 | 9 | 18% | 19.6% |
| Controls | 2 | 4 | 2 | 4 | 1 | 11 | 22% | |
| | 3 | 4 | 2 | 3 | 0 | 9 | 18% | |
| | 6 | 7 | 0 | 1 | 0 | 8 | 16% | |
| | 7 | 7 | 2 | 3 | 0 | 12 | 24% | |
| Canine | 43 | 1 | 0 | 0 | 0 | 1 | 2% | 15.2% |
| Mice | 44 | 8 | 2 | 3 | 1 | 14 | 28% | |
| | 45 | 1 | 0 | 1 | 0 | 2 | 4% | |
| | 46 | 3 | 0 | 7 | 1 | 11 | 22% | |
| | 47 | 3 | 4 | 2 | 1 | 10 | 20% | |

Due to low numbers of CD34⁺ stem cells that are available from canine cord blood, we attempted to isolate stem cells from bone marrow collected from newborn puppies. CD34⁺ stem cells were isolated from bone marrow using a similar method to the cord blood isolation protocol. Using bone marrow from three mixed donor dogs, and following MACS beads and column isolation, we were able to isolate 4.4×10⁵ CD34⁺ cells. These cells were engrafted into 7 NSG mice. At 12 weeks post engraftment the mice had canine CD45⁺ cell levels of 21±19% of the total lymphocyte/granulocyte population.

CnMice of bone marrow CD34⁺ cell origin were then infected with 100 JYD L3 Di. These mice were necropsied on Days 40 through 42 post-infection. The recovery data is shown in Table 26. No significant difference was seen between the two groups. The absence of significant difference in parasite recoveries between the controls and either CnMouse model suggests these are viable models to begin to understand the relationship between the canine immune system and the development of D. immitis.

TABLE 26
Summary data and recovery of Di for CnMice and NSG control
mice infected with FR3 Di.

| | | | U. | | | | % | Average % |
|---|---|---|---|---|---|---|---|---|
| Group | Mouse # | Skin | Muscle | L. Muscle | Pluck | Total | Recovery | Recovery |
| Control | 1 | 18 | 9 | 10 | 0 | 37 | 37% | 33.1% |
| NSG | 2 | 9 | 10 | 9 | 0 | 28 | 28% | |
| Mice | 3 | 8 | 7 | 19 | 0 | 34 | 34% | |
| | 4 | 11 | 3 | 8 | 0 | 22 | 22% | |
| | 5 | 20 | 4 | 15 | 1 | 40 | 40% | |
| | 6 | 14 | 4 | 19 | 0 | 37 | 37% | |
| | 7 | 7 | 5 | 13 | 0 | 25 | 25% | |
| | 8 | 10 | 18 | 10 | 0 | 38 | 38% | |
| | 9 | 12 | 5 | 11 | 0 | 28 | 28% | |
| | 10 | 18 | 7 | 17 | 0 | 42 | 42% | |
| CnMice | 11 | 11 | 10 | 12 | 5 | 38 | 38% | 36.3% |
| | 12 | 14 | 6 | 1 | 3 | 24 | 24% | |
| | 13 | 25 | 9 | 7 | 0 | 41 | 41% | |
| | 14 | 12 | 6 | 15 | 1 | 34 | 34% | |
| | 15 | 22 | 18 | 21 | 0 | 61 | 61% | |
| | 16 | 13 | 2 | 6 | 1 | 22 | 22% | |
| | 17 | 15 | 7 | 11 | 1 | 34 | 34% | |

### Example 4—Development and Validation of an In Vivo Model of Gastrointestinal Nematode Infection

Haemonchus contortus is a gastrointestinal worm that infects mainly sheep and goats, but also cattle. These worms do not affect dogs and cats. The disease caused by Haemonchus worms is called haemonchosis. Haemonchus worms have a direct life cycle (there are no intermediate hosts involved). Adult females lay eggs in the stomach of the host that are shed with the feces. Once in the environment, the eggs release the L1-larvae that complete development to infective L3-larvae in about 4 to 7 days. Livestock becomes infected after ingesting infective larvae. These larvae enter the pit of gastric glands and feed on blood flowing out of the lesions they cause to the stomach's lining. Later, they complete development to adult worms and females start producing eggs.

In a pilot experiment, mice were infected with either 500 or 1,000 larvae of H. contortus. In addition, some of the mice were further immune compromised by treatment with the steroid methylprednisolone acetate. Following infection, mice showed a drop in weight of 10-20%, and then either regained or remained the same throughout the rest of the experiment. Mice were necropsied periodically over 8 weeks, looking in the stomach for adult worms and in the intestinal contents for eggs. It was discovered that H. contortus larvae develop to adults that contained blood within their intestines. Importantly, eggs were seen in the intestinal contents at 28 days post-infection. Steroid treatment to further compromise the immune response did not increase worm recoveries.

NSG mice were pre-treated with methylprednisolone acetate (MPA) one week prior to infection. H. contortus L3 were both agar cleaned and/or cleaned with NCTC/IMDM (NI) media with antibiotics. 500 and 1000 L3 were dosed to control and MPA-treated mice by oral gavage. At dosing, mice were weighed and given a general health score. Mice were checked biweekly for health scores and weighed once a week at MPA dosing (MPA injections are weekly). Forty total mice were infected: 20 with MPA and 20 controls, with 10 in each group for doses of 500 and 1000.

The day prior to necropsy, mice were fasted. Stomachs and small intestines were then removed. The stomach was cut open and hung in a 40 mL conical in PBS. The small intestines were cut open and hung around a paper clip in a 50 mL conical in PBS. The tissues were then incubated at 50° C. for 2.5 hours, after which the tissues were discarded. The tubes were shaken and poured through a 200 μm mesh strainer and rinsed. Worms were collected by back washing off of the strainer and were counted. Worms were saved fixed with glycerol/alcohol to be measured.

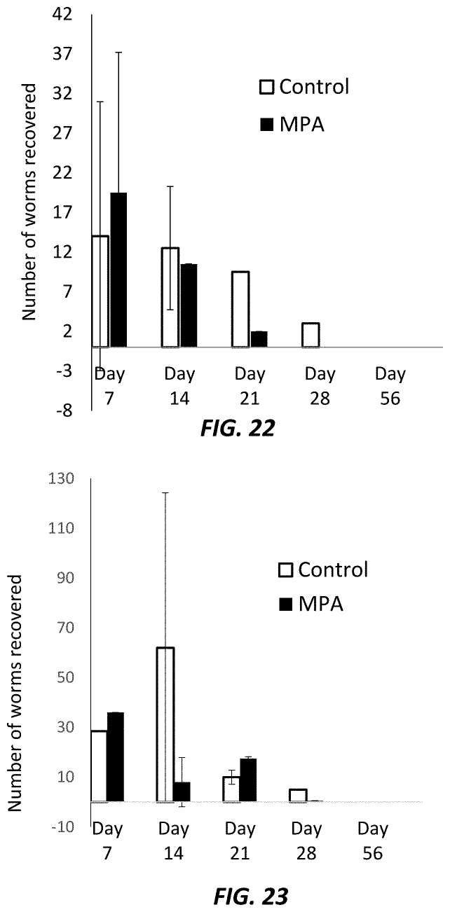

H. contortus recovery counts for the 500 and 1000 starting infections are shown in Tables 27 and 28, respectively, and in FIGS. 22 and 23, respectively. “Recovery” refers to the number of He worms recovered from a mouse. Eggs collected from fecal content between Days 23-31 post-infection (day 28 post-infection) in four of the infected mice are shown in Table 29.

TABLE 27
Hc recovery counts from mice with starting infection of 500 Hc L3.

| | Control | | | MPA | | |
|---|---|---|---|---|---|---|
| | Mouse | Re- | | Mouse | | |
| | ID | covery | Average | ID | Recovery | Average |
| 7 days PI | 7 | 26 | 14 | 1 | 36 | 19.5 |
| | 8 | 2 | | 3 | 3 | |
| 14 Days PI | 15 | 0 | 12.5 | 12 | 2 | 10.5 |
| | 16 | 25 | | 11 | 19 | |
| 21 Days PI | 22 | 4 | 9.5 | 18 | 2 | 2 |
| | 23 | 15 | | 19 | 2 | |
| 28 Days PI | 30 | 3 | 3 | 25 | 0 | 0 |
| | 31* | 3 | | 27 | 0 | |
| 56 Days PI | 39 | 0 | 0 | 33 | 0 | 0 |
| | 40 | 0 | | 34 | 0 | |

*indicates egg collection

TABLE 28
Hc recovery counts from mice with starting infection of 1000 Hc L3.

| | Control | | | MPA | | |
|---|---|---|---|---|---|---|
| | Mouse | Re- | | Mouse | | |
| | ID | covery | Average | ID | Recovery | Average |
| 7 days PI | 5 | 33 | 28.5 | 2 | 24 | 36 |
| | 6 | 24 | | 4 | 48 | |
| 14 Days PI | 13 | 18 | 62 | 10 | 1 | 8 |
| | 14 | 106 | | 9 | 15 | |
| 21 Days PI | 21 | 11 | 10 | 17 | 15 | 17.5 |
| | 24 | 9 | | 20 | 20 | |
| 28 Days PI | 29* | 7 | 5 | 26* | 0 | 0.5 |
| | 32* | 3 | | 28 | 1 | |
| 56 Days PI | 37 | 0 | 0 | 35 | 0 | 0 |
| | 38 | 0 | | 36 | 0 | |

*indicates egg collection

TABLE 29
Eggs found at week four in four mice infected with either
500 or 1000 Hc L3.

| Mouse ID | Experiment | # Eggs Found |
|---|---|---|
| 26 | 1000 infected, MPA | 2 |
| 29 | 1000 infected, control | 9 |
| 31 | 500 infected, control | 41 |
| 32 | 1000 infected, control | 8 |

Mice were checked biweekly over the infection period for a visual health screen and always scored 0 on our health assessment (score of 8 is an indicator for euthanasia). Mice were weighed weekly to monitor for excessive weight loss (data not shown).

In a second experiment, 43 NSG mice were infected with 1000 L3 as described above. H. contortus recovery counts at various time points are shown in Table 30. Eggs were recovered from the mice 26 days post-infection and were incubated at 26° C. for 18 days. Twenty L1 larvae were recovered.

TABLE 30
Hc recovery counts from mice with starting infection of 1000 Hc L3.

| Time Point | Number of Mice Infected | Average Recovery |
|---|---|---|
| 15 Days PI | 6 | 15 |
| 23 Days PI | 6 | 4 |
| 28 Days PI | 22 | 2 |
| 31 Days PI | 8 | 1 |

Strongyloides stercoralis (threadworm) is a human pathogenic parasitic roundworm that causes the disease strongyloidiasis. The adult parasitic stage lives in tunnels in the mucosa of the small intestine. S. stercoralis has been reported in other mammals, including cats and dogs. S. stercoralis infects an estimated 100 million people. Its ability to autoinfect its host enables it to persist for decades undetected and to progress to a potentially fatal hyperinfection that often is induced by glucocorticoid treatment. The mouse model described herein recapitulates all forms of human strongyloidiasis. Treatment with methylprednisolone acetate (MPA), a synthetic glucocorticoid, induces hyperinfection in NSG mice.

NSG mice infected by subcutaneous injection with 5000 S. stercoralis L3 larvae, either with or without MPA. Mice were treated with 2.5 mg/kg EMO or 3 mg/kg IVM on days 1, 15, and 30 post-injection. Adult worms, L1 larvae, or L3 larvae were counted in the mice at 6 weeks post-injection. As shown in Tables 31 and 32, EMO and IVM clearly controlled the intestinal infection, eliminating adult worms and L1 larvae, respectively, in models of both normal, chronic infection (control) and hyperinfection (MPA). As shown in Table 33, EVO and IVM reduced the circulating L3 in models of both normal, chronic infection (control) and hyperinfection (MPA), but there were still some circulating L3 in the body.

TABLE 31
Adult S. stercoralis recovery counts from mice
at 6 weeks post-injection.

| | | | EMO | | | |
|---|---|---|---|---|---|---|
| | Control | MPA | Control | EMO/MPA | IVM Control | IVM/MPA |
| | 16 | 437 | 0 | 0 | 0 | 0 |
| | 2 | 581 | 0 | 0 | 0 | 0 |
| | 17 | 447 | 0 | 0 | 0 | 0 |
| | 10 | 888 | 0 | 0 | 0 | 0 |
| | 10 | 235 | 1 | 0 | 0 | 0 |
| | | 34 | | 0 | | 0 |
| | | | | 0 | | 0 |
| | | | | | | 0 |
| mean | 11 | 437 | 0.2 | 0 | 0 | 0 |
| SD | 6 | 292 | 0.4 | 0 | 0.0 | 0 |

TABLE 32
L1 S. stercoralis recovery counts from mice at 6 weeks post-injection.

| | | | EMO | | | |
|---|---|---|---|---|---|---|
| | Control | MPA | Control | EMO/MPA | IVM Control | IVM/MPA |
| | 32 | 8456 | 0 | 0 | 0 | 0 |
| | 4 | 9181 | 0 | 0 | 0 | 0 |
| | 15 | 3464 | 0 | 0 | 0 | 0 |
| | 46 | 670 | 0 | 0 | 0 | 0 |
| | 64 | 86 | 0 | 0 | 0 | 0 |
| | | 24 | | 0 | | 0 |
| | | | | 0 | | 0 |
| | | | | | | 0 |
| mean | 32 | 3647 | 0 | 0 | 0 | 0 |
| SD | 24 | 4206 | 0.0 | 0 | 0.0 | 0 |

TABLE 33
L3 S. stercoralis recovery counts from mice at 6 weeks post-injection.

| | | | EMO | | | |
|---|---|---|---|---|---|---|
| | Control | MPA | Control | EMO/MPA | IVM Control | IVM/MPA |
| | 496 | 2760 | 8 | 102 | 9 | 25 |
| | 202 | 1766 | 0 | 130 | 26 | 16 |
| | 218 | 632 | 1 | 39 | 210 | 40 |
| | 407 | 1015 | 5 | 18 | 2 | 38 |
| | 497 | 275 | 5 | 4 | 6 | 19 |
| | | 31 | | 123 | | 24 |
| | | | | 228 | | 4 |
| | | | | | | 13 |
| mean | 364 | 1080 | 4 | 92 | 51 | 22 |
| SD | 145 | 1024 | 3 | 79 | 90 | 12 |

1-39. (canceled)

40. A genetically engineered immunocompromised rodent, wherein: the immunocompromised rodent has been genetically engineered to disrupt or delete genes required for wild type immune function; and wherein the immunocompromised rodent is characterized by absence of mature T and B cells and lack of functional NK cells; and/or by a deficiency in cytokine signaling and multiple defects in innate immunity; and/or by absence of production of detectable serum immunoglobulin.

41. The genetically engineered immunocompromised rodent of claim 40, wherein: the Prkdc gene of the immunocompromised rodent comprises a severe combined immunodeficiency (scid) mutation (Prkdc^{scid}).

42. The genetically engineered immunocompromised rodent of claim 40, wherein: the rodent is a mouse, preferably an NOD scid IL2 receptor gamma chain knockout mouse (NSG) (NOD-SCID-IL2Ry −/−) or an NRG (NOD-Rag1^{null}IL2rg^{null}) immunodeficient mouse.

43. The genetically engineered immunocompromised rodent of claim 40, wherein: the Prkdc gene of the immunocompromised rodent comprises a severe combined immunodeficiency (scid) mutation (Prkdc^{scid}), and wherein the rodent is a mouse, preferably an NOD scid IL2 receptor gamma chain knockout mouse (NSG) (NOD-SCID-IL2Ry −/−) or an NRG (NOD-Rag1^{null}IL2rg^{null}) immunodeficient mouse.

44. The genetically engineered immunocompromised rodent of claim 40, wherein the immunocompromised rodent does not comprise engrafted immune cells from a different species.

45. The genetically engineered immunocompromised rodent of claim 40, wherein the immunocompromised rodent comprises engrafted immune cells from a definitive host of the parasitic worm, optionally wherein the engrafted immune cells are canine immune cells.

46. The genetically engineered immunocompromised rodent of claim 40, wherein the immunocompromised rodent is infected with one or more adult parasitic worms.

47. The genetically engineered immunocompromised rodent of claim 40, wherein the immunocompromised rodent is infected with one or more adult parasitic worms, and wherein at least one of the parasitic worms is a nematode.

48. The genetically engineered immunocompromised rodent of claim 40, wherein the immunocompromised rodent is infected with one or more adult parasitic worms, and wherein at least one of the parasitic worms is a filarial worm, optionally wherein the filarial worm is Dirofilaria immitis.

49. The genetically engineered immunocompromised rodent of claim 40, wherein the immunocompromised rodent is infected with one or more adult parasitic worms, and wherein at least one of the parasitic worms is a gastrointestinal parasitic worm, optionally wherein the gastrointestinal parasitic worm is Strongyloides stercoralis or Haemonchus contortus.

50. The genetically engineered immunocompromised rodent of claim 40, wherein the immunocompromised rodent comprises engrafted canine immune cells, and wherein the immunocompromised rodent is infected with Dirofilaria immitis.

51. A method for determining the effectiveness of a compound or combination of compounds for treating or preventing a parasitic worm infection, comprising: infecting the immunocompromised rodent of claim 40 with a specific number of parasitic worm larvae, and treating the infected rodent with the compound or combination of compounds to be tested; determining the number of parasitic worm larvae, parasitic worms, and/or adult parasitic worms present in treated, infected rodent tissue samples; and comparing the number of parasitic worm larvae, parasitic worms, and/or adult parasitic worms present in untreated, infected control rodent tissue samples.

52. The method of claim 51, wherein the immunocompromised rodent tissue samples are collected from a necropsied immunocompromised rodent.

53. The method of claim 51, wherein the parasitic worm infecting the immunocompromised rodent is a nematode.

54. The method of claim 51 wherein the parasitic worm infecting the immunocompromised rodent is a filarial worm, optionally wherein the filarial worm is Dirofilaria immitis, or a gastrointestinal parasitic worm, optionally wherein the gastrointestinal parasitic worm is Strongyloides stercoralis or Haemonchus contortus.

55. The method of claim 51, wherein the parasitic worm larvae are selected from the group consisting of microfilariae, L3 larvae, and L4 larvae.

56. The method of claim 51, further comprising comparing the parasiticidal efficacy of the compound or combination of compounds against the parasiticidal efficacy of either or both a macrocyclic lactone (ML) parasiticidal compound and/or a depsipeptide parasiticidal compound.

57. The method of claim 56, wherein the ML compound is selected from avermectin, ivermectin, doramectin, abamectin, milbemycin and moxidectin.

58. The method of claim 56, wherein the depsipeptide parasiticidal compound is emodepside.
